(19) United States
(12) Patent Application Publication
(10) Pub. No.: US 2018/0232537 A1
(43) Pub. Date: Aug. 16, 2018

(54) INFORMATION PROCESSING APPARATUS AND INFORMATION PROCESSING METHOD

(71) Applicant: FUJITSU LIMITED, Kawasaki-shi (JP)

(72) Inventors: Ayako Masuda, Yokohama (JP); Akito YAMAZAKI, Kawasaki (JP); Kazunori Kobashi, Yamato (JP)

(73) Assignee: FUJITSU LIMITED, Kawasaki-shi (JP)

(21) Appl. No.: 15/868,266

(22) Filed: Jan. 11, 2018

(30) Foreign Application Priority Data
Feb. 10, 2017 (JP) 2017-022918

(51) Int. Cl. G06F 21/62; G16H 50/70; G16H 10/60

(52) U.S. Cl. CPC G06F 21/6254; G16H 10/60; G16H 50/70

(57) ABSTRACT

An information processing apparatus includes a memory and a processor coupled to the memory and configured to: convert personal information of a first format, which is stored in a storage device and is used by one of a plurality of information providing institutions, into personal information of a second format used by an analysis device which analyzes personal information collected from the plurality of information providing institutions; conceal the personal information of the second format to generate concealed personal information of the second format; and transmit the concealed personal information of the second format to the analysis device.

### CROSS-REFERENCE TO RELATED APPLICATION

This application is based upon and claims the benefit of priority of the prior Japanese Patent Application No. 2017-022918, filed on Feb. 10, 2017, the entire contents of which are incorporated herein by reference.

### FIELD

The embodiments discussed herein are related to an information processing apparatus, an information processing method, and a non-transitory computer-readable recording medium having stored therein a program.

### BACKGROUND

In recent years, the need for big data analysis is increasing more and more. For the big data analysis, it is desirable to collect data samples as many as possible in order to obtain more accurate and useful analysis results.

There may be a plan on a government level to implement a future policy to promote big data analysis in the domestic medical field. According to this policy, data on hospital electronic medical record are collected and are processed into anonymous data which are to be provided to organizations that wish to utilize the anonymous data as data available for the big data analysis.

The electronic medical record is data containing a lot of personal information related to patient's privacy. For this reason, when collecting such data in large quantities, it is desirable to take measures to prevent the leakage of personal information.

A technique that enables analysis processing and statistical processing of patient data or the like while protecting the personal privacy related to personal data such as patient data is known.

Related technologies are disclosed in, for example, Japanese Laid-Open Patent Publication No. 2003-005645.

### SUMMARY

According to an aspect of the embodiments, an information processing apparatus is provided including a memory and a processor couple to the memory and configured to: convert personal information of a first format, which is stored in a storage device and is used by one of a plurality of information providing institutions, into personal information of a second format used by an analysis device which analyzes personal information collected from the plurality of information providing institutions; conceal the personal information of the second format to generate concealed personal information of the second format; and transmit the concealed personal information of the second format to the analysis device.

The object and advantages of the invention will be realized and attained by means of the elements and combinations particularly pointed out in the claims. It is to be understood that both the foregoing general description and the following detailed description are exemplary and explanatory and are not restrictive of the invention, as claimed.

### BRIEF DESCRIPTION OF DRAWINGS

FIG. 1 is a view illustrating the configuration of an information processing system according to an embodiment;

FIG. 2 is an example of a raw electronic medical record information DB of an existing system;

FIG. 3 is an example of a raw electronic medical record information DB of a new system;

FIG. 4 is an example of a correspondence relation table;

FIG. 5 is a view illustrating items to be concealed and a concealment method;

FIG. 6 is an example of a concealment mapping table;

FIG. 7 is an example of a concealment electronic record information DB;

FIG. 8 is an example of a concealment logic distribution management table;

FIG. 9 is an example of an automatic conversion processing mechanism list;

FIGS. 10A and 10B are sequence diagrams of an installation process of an electronic medical record installation set according to the embodiment;

FIG. 11 is a view illustrating an installation information input screen;

FIG. 12 is a view illustrating an installation information input screen;

FIGS. 13A and 13B are detailed sequence diagrams of an electronic medical record format conversion process;

FIGS. 14A and 14B are sequence diagrams of an electronic medical record input process according to an embodiment;

FIG. 15 is a view illustrating an example of an electronic medical record input screen;

FIG. 16 is a sequence diagram of a data transfer process according to an embodiment;

FIG. 17 is a sequence diagram of a data analysis process according to an embodiment;

FIG. 18 is a view illustrating an example of a concealment electronic medical record analysis process;

FIG. 19 is a sequence diagram of a raw electronic medical record restoration process according to an embodiment;

FIG. 20 is a sequence diagram of a concealment electronic medical record and concealment mapping table restoration process according to an embodiment;

FIG. 21 is a detailed sequence diagram of a restoration process in case where only the concealed electronic medical record information DB fails;

FIG. 22 is a detailed sequence diagram of a restoration process in a case where only the concealment mapping table fails;

FIG. 23 is a detailed sequence diagram of a restoration process in a case where both of the concealed electronic medical record information DB and the concealment mapping table fail; and

FIG. 24 is a view illustrating the configuration of an information processing apparatus (computer).

### DESCRIPTION OF EMBODIMENTS

Based on the above-mentioned policy of the government, when an information analysis institution collects and analyzes electronic medical record from various hospitals, when the formats of the electronic medical record of the hospitals are different from each other, the information analysis institution arranges and then analyses the formats of the electronic medical record. However, such a process of arrangement of the formats by the information analysis institution is a troublesome and time-consuming task.

Without being limited to analyzing electronic medical record provided from hospitals, the same is true for analyzing other information provided from other information providing institutions.

Hereinafter, embodiments will be described with reference to the drawings.

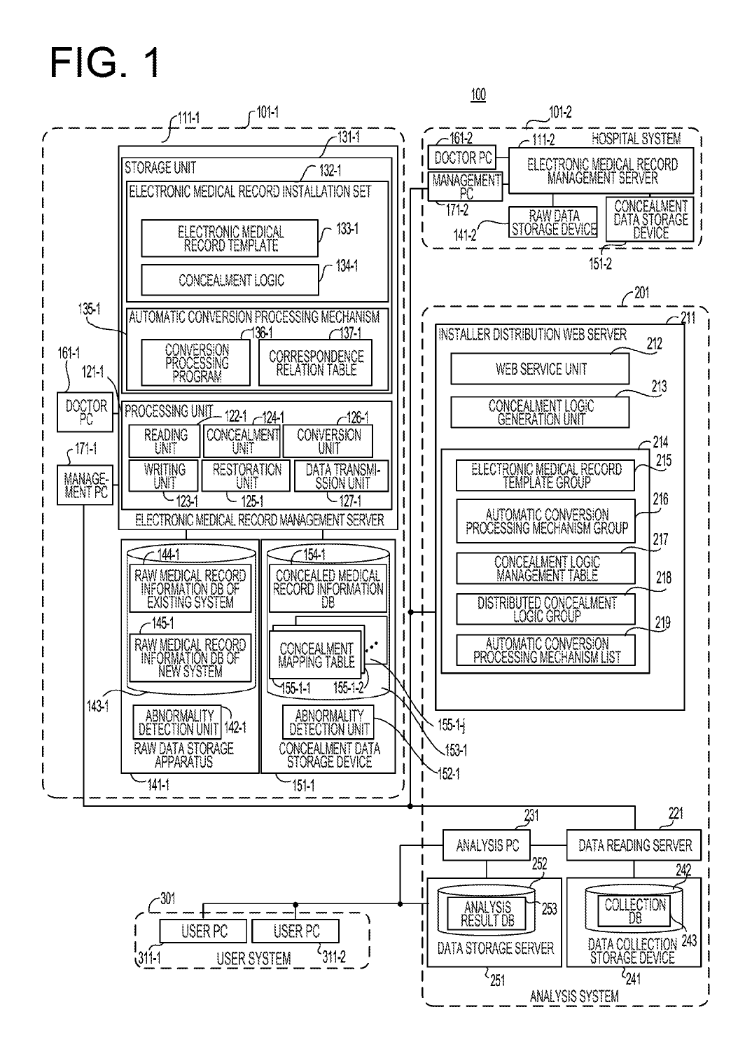

FIG. 1 is a view illustrating the configuration of an information processing system according to an embodiment.

In the information processing system 100 of FIG. 1, an information providing institution is a hospital that provides data of electronic medical record, an information analysis institution is an institution of the government or the like that collects and analyzes the data of electronic medical record, and an information utilization institution is an institution that utilizes results of the analysis, such as a research institution, a pharmaceutical company, etc. The electronic medical record is an example of personal information.

The information processing system 100 includes a hospital system 101-1 of a hospital A, a hospital system 101-2 of a hospital B, an analysis system 201 of an information analysis institution, and a user system 301 of an information utilization institution. The number of hospital systems is not limited to two but may be three or more when the number of hospitals is three or more. For example, a plurality of hospitals existing nationwide may be information providing institutions.

A hospital system 101-i (i=1, 2) includes an electronic medical record management server 111-i, a raw data storage device 141-i, a concealment data storage device 151-i, a doctor personal computer (PC) 161-i and a management PC 171-i. The hospital system 101-1 stores and manages electronic medical record of patients at the hospital A and the hospital system 101-2 stores and manages electronic medical record of patients at the hospital B.

An electronic medical record management server 111-1 includes a processing unit 121-1 and a storage unit 131-1. The electronic medical record management server 111-1 is connected to and can communicate with a raw data storage device 141-1, a concealment data storage device 151-1, a doctor PC 161-1 and a management PC 171-1 via a network.

The processing unit 121-1 includes a reading unit 122-1, a writing unit 123-1, a concealment unit 124-1, a restoration unit 125-1, a conversion unit 126-1 and a data transmission unit 127-1.

The reading unit 122-1 reads electronic medical record included in a raw medical record information DB 144-1 of an existing system and electronic medical record included in a raw medical record information DB 145-1 of a new system.

The writing unit 123-1 writes electronic medical record in the raw medical record information DB 145-1 of the new system.

The concealment unit 124-1 conceals electronic medical record. Specifically, the concealment unit 124-1 conceals information on predetermined some of items included in the electronic medical record.

The restoration unit 125-1 restores the raw medical record information DB 145-1, a concealed medical record information DB 154-1 and a concealment mapping table 155-1-j.

The conversion unit 126-1 converts electronic medical record of different formats of the raw medical record information DB 144-1 of the existing system into electronic medical record of a common format to generate the raw medical record information DB 145-1. The common format is a format of an electronic medical record analyzed by an analysis PC 231 and is a format common for electronic medical record used in various hospitals.

The data transmission unit 127-1 transmits the concealed medical record information DB 154-1. The data transmission unit 127-1 is an example of a transmission unit.

The storage unit 131-1 is a storage device that stores programs, data and the like used in the electronic medical record management server 111-1. The storage unit 131-1 stores an electronic medical record installation set 132-1.

The electronic medical record installation set 132-1 includes an electronic medical record template 133-1, a concealment logic 134-1 and an automatic conversion processing mechanism 135-1. An electronic medical record installation set 132-1 suitable for the configuration (type and version of an operating system and type of a CPU) of the electronic medical record management server 121-1 is downloaded from an installer distribution web server 211.

The electronic medical record template 133-1 is a program for generating, writing and reading electronic medical record of the common format common to various hospitals.

The concealment logic 134-1 is a program for concealing data of predetermined items of the electronic medical record. The items concealed by the concealment logic 134-1 and a concealment method may be different from the concealment logics of other hospitals. It is desirable that the same concealment logic be used in hospitals of the same series (group).

The automatic conversion processing mechanism 135-1 includes a conversion processing program 136-1 and a correspondence relation table 137-1.

The conversion processing program 136-1 is a program that converts the electronic medical record of the format of the existing system into the electronic medical record of the format of the new system. The format of the electronic medical record of the new system is the format of the electronic medical record generated by the electronic medical record template 132-1. In addition, the format of the electronic medical record of the new system is the common format which is common for each hospital.

The correspondence relation table 137-1 indicates where in the raw medical record information DB 144-1 of the existing system the data of each item of the raw medical record information DB 145-1 of the new system is located.

Details of the correspondence relation table 137-1 will be described later.

The raw data storage device 141-1 includes an abnormality detection unit 142-1 and a storage unit 143-1.

The abnormality detection unit 142-1 detects an abnormality of the raw medical record information DB 145-1 stored in the storage unit 143-1. That is, the abnormality detection unit 142-1 detects the effect that an electronic medical record included in the raw medical record information DB 145-1 is damaged and cannot be read. The abnormality detection unit 142-1 regularly checks whether or not the electronic medical record included in the raw medical record information DB 145-1 are normal, that is, whether or not the electronic medical record included in the raw medical record information DB 145-1 can be read out.

The storage unit 143-1 stores the raw medical record information DB 144-1 of the existing system and the raw medical record information DB 145-1 of the new system.

The raw medical record information DB 144-1 of the existing system includes electronic medical record of another format created by a previously used system. The electronic medical record included in the raw medical record information DB 144-1 of the existing system is not concealed.

The raw medical record information DB 145-1 of the new system includes electronic medical record of the common format created by the electronic medical record installation set 132-1.

The concealment data storage apparatus 151-1 includes an abnormality detection unit 152-1 and a storage part 153-1.

The abnormality detection unit 152-1 detects an abnormality of the concealed medical record information DB 154-1 and the concealment mapping table 155-1-j (j=1 to n: n is the maximum value of patient IDs included in the concealed medical record information DB 154-1, that is, the number of patients recorded in the concealed medical record information DB 154-1). That is, the abnormality detection unit 152-1 detects the effect that the electronic medical record included in the concealed medical record information DB 154-1 and the concealment mapping table 155-1-j are damaged and cannot be read out. The abnormality detection unit 152-1 regularly checks whether or not the electronic medical record included in the concealed medical record information DB 154-1 and the concealment mapping table 155-1-j are normal, that is, whether or not the electronic medical record included in the concealed medical record information DB 154-1 and the concealment mapping table 155-1-j can be read out.

The storage unit 153-1 stores the concealed medical record information DB 154-1 and the concealment mapping table 155-1-j.

The concealed medical record information DB 154-1 includes concealment electronic medical record obtained by concealing the electronic medical record included in the raw medical record information DB 145-1.

The information of items to be concealed of pre-concealed electronic medical record of patients and the information of items to be concealed of post-concealed electronic medical record of patients are recorded in the concealment mapping table 155-1-j in association. The information of items to be concealed of pre-concealed electronic medical record of patients corresponding to a patient ID=j and the information of items to be concealed of post-concealed electronic medical record of the patients are recorded in the concealment mapping table 155-1-j.

The doctor PC 161-1 is a device used by a doctor to input information on an electronic medical chart.

The management PC 171-1 performs processes such as sending an instruction to the electronic medical record management server 111-1 and transmission of the concealed electronic medical record to the analysis system 201

An electronic medical record management server 111-2, a raw data storage device 141-2, a concealment data storage device 151-2, a doctor PC 161-2, and a management PC 171-2 have the same functions as the electronic medical record management server 111-1, the raw data storage device 141-1, the concealment data storage device 151-1, the doctor PC 161-1 and the management PC 171-1 and therefore, detailed explanation of which will not be repeated for the purpose of brevity.

The analysis system 201 includes an installer distribution web server 211, a data reading server 221, an analysis PC 231, a data collection storage device 241 and a data storage server 251.

The installer distribution web server 211 includes a web service unit 212, a concealment logic generation unit 213 and a storage unit 214.

In response to a request from the management PC 171, the web service unit 212 creates and distributes an electronic medical record installation set.

The concealment logic generation unit 213 generates concealment logic. The concealment logic is a program for concealing data. Items to be concealed of electronic medical record and concealment methods may be different for different generated concealment logics.

The storage unit 214 stores an electronic medical record template group 215, an automatic conversion processing mechanism group 216, a concealment logic management table 217, a distributed concealment logic group 218 and an automatic conversion processing mechanism list 219.

The electronic medical record template group 215 is a set of electronic medical record templates for creating electronic medical record of the common format common for hospitals. The electronic medical record template group 215 is a set of plural electronic medical record templates that operate with the type and version of an OS and the type of a CPU.

The automatic conversion processing mechanism group 216 is a set of automatic conversion processing mechanisms for converting electronic medical record of other formats to create electronic medical record of the common format. The automatic conversion processing mechanism group 216 is a set of plural automatic conversion processing mechanisms, each of which converts an electronic medical record of one of plural formats, which are generated by plural products of plural electronic medical record makers that generate electronic medical record, into electronic medical record of the common format. For example, among the plural automatic conversion processing mechanisms, an automatic conversion processing mechanism H-1 converts an electronic chart of a first format generated by an electronic medical record system for clinic, which is a product of an electronic medical record maker H company, into an electronic medical record of the common format. Among the plural automatic conversion processing mechanisms, an automatic conversion processing mechanism H-2 converts an electronic chart of a second format generated by an electronic medical record system for ophthalmologic clinic, which is a product of the electronic medical record maker H company, into an electronic medical record of the common format. In this way, the automatic conversion processing mechanism group 216 includes plural automatic conversion processing mechanisms corresponding to formats generated by plural electronic medical record makers and plural products.

The concealment logic management table 217 is a table in which information indicating the concealment logic and distribution destinations of the concealment logic are recorded in association.

The distributed concealment logic group 218 is a set of concealment logics distributed to the hospital system 101-i.

The automatic conversion processing mechanism list 219 is a list of automatic conversion processing mechanisms included in the automatic conversion processing mechanism group 216.

The data reading server 221 receives concealment electronic medical record from each hospital system 101-i and writes them in a collection DB 243 of the data collection storage device 241.

The analysis PC 231 reads the concealment electronic medical record included in the collection DB 243 from the data collection storage device 241, analyzes the read concealment electronic medical record, and writes the analysis result in an analysis result DB 253 of the data storage server 251.

The data collection storage device 241 includes a storage unit 242. The storage unit 242 stores the collection DB 243. The collection DB 243 includes concealment electronic medical record acquired from each hospital system 101-i.

The data storage server 251 includes a storage unit 252. The storage unit 252 stores the analysis result DB 253. The analysis result DB 253 includes a result of the analysis of the concealment electronic medical record by the analysis PC 231.

The user system 301 includes a user PC 311-i. A user of an information utilization institution uses the user PC 311-I to request the analysis system 201 to provide the analysis result and acquires the analysis result from the analysis result DB 253.

It is to be noted that the configuration of the information processing system 100 described above is merely an example and may be appropriately changed. For example, the raw data storage device 141-1 and the concealment data storage device 151-1 may be the same device. In addition, the raw data storage device 141-1 and the concealment data storage device 151-1 may be located in the electronic medical record management server 111-1.

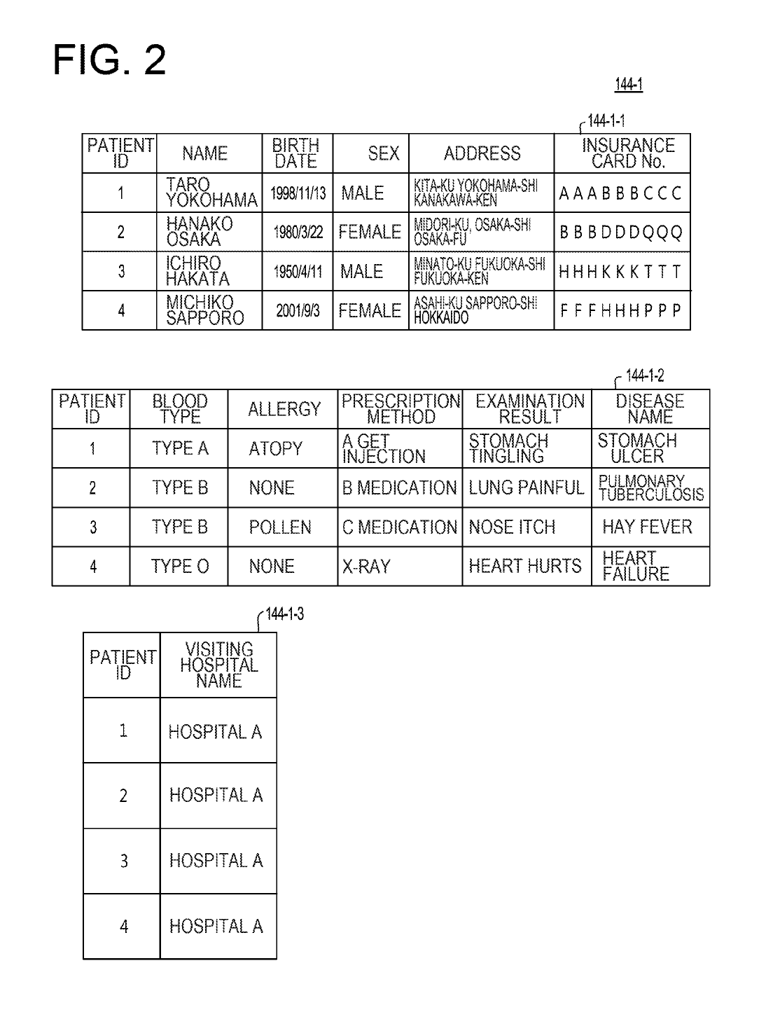

FIG. 2 illustrates an example of the raw electronic medical record information DB of the existing system.

The raw electronic medical record information DB 144-1 of the existing system includes a patient information table 144-1-1, a diagnosis information table 144-1-2 and a reception hospital record 144-1-3. In the raw electronic medical record information DB 144-1 of the existing system, electronic medical record are recorded in a format different from the format of the electronic medical record analyzed by the analysis PC 231.

The patient information table 144-1-1 includes, as items, a patient ID, a name, a birth date, a sex, an address and an insurance card number.

The diagnosis information table 144-1-2 includes, as items, a patient ID, a blood type, an allergy, a prescription result, an examination result and a disease name.

The reception hospital record 144-1-3 includes, as items, a patient ID and a reception hospital name.

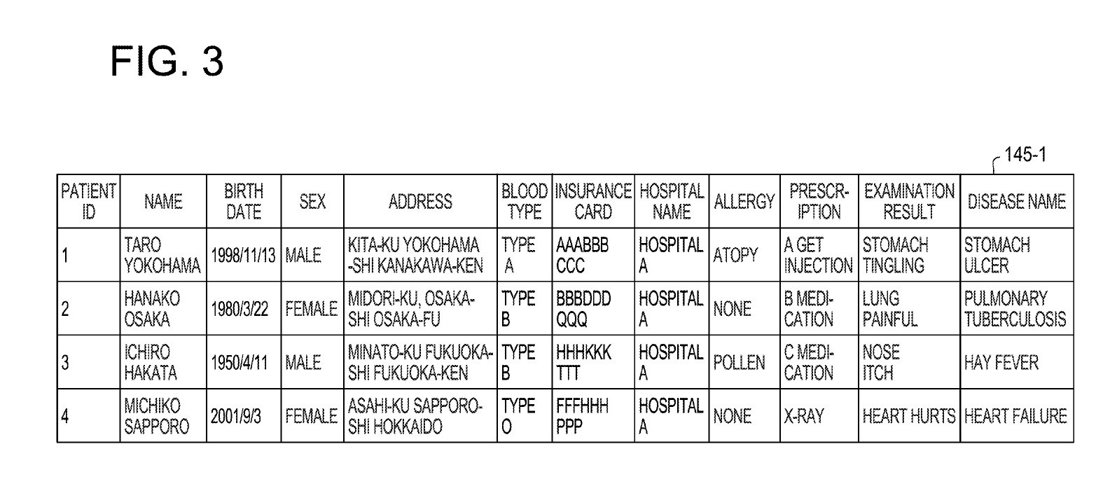

FIG. 3 illustrates an example of the raw electronic medical record information DB of the new system.

The raw electronic medical record information DB 145-1 of the new system includes, as items, a patient ID, a name, a birth date, a sex, an address, a blood type, an insurance card ID, a hospital name, an allergy, a prescription, an examination result and a disease name. The raw medical record information DB 145-1 of the new system includes items of electronic medical record of a format of electronic medical record analyzed by the analysis PC 231 and of the common format common for hospitals. That is, in the raw medical record information DB 145-1 of the new system, the electronic medical record is recorded in the common format.

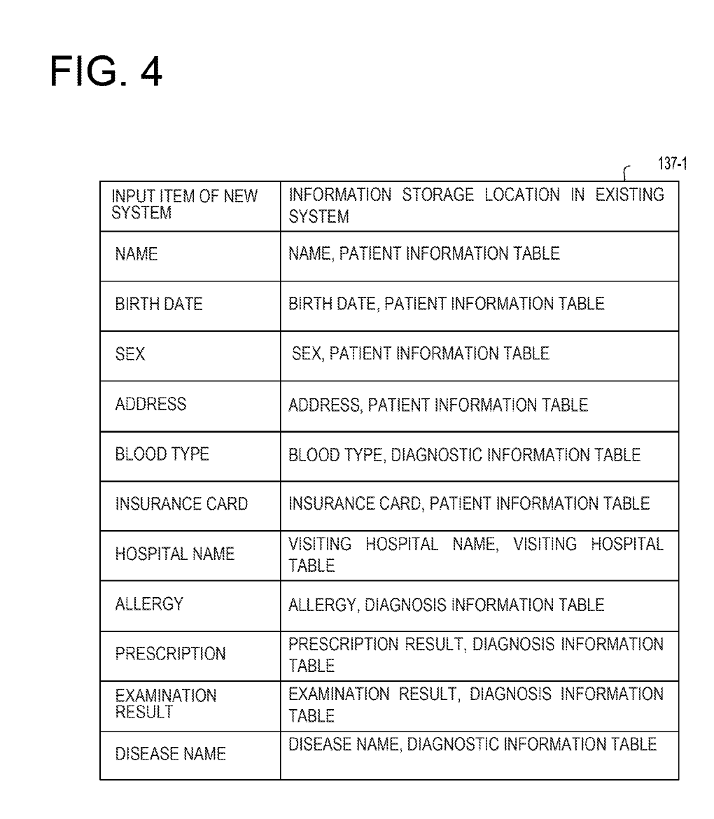

FIG. 4 illustrates shows an example of a correspondence relation table.

The correspondence relation table 137-1 includes, as items, a new system input item and an information storage location in the existing system, which are in recorded in association.

The new system input item indicates the items of the raw medical record information DB 145-1 of the new system. That is, the items of electronic medical record of the common format common for hospitals are shown.

The information storage location in the existing system indicates locations of data in the raw electronic medical record information DB 144-1 of the existing system corresponding to the new system input item.

For example, the first row of the correspondence relation table 137-1 of FIG. 4 indicates that the data of the “name” of the input item of the new system corresponds to the data of “name” of the patient information table 144-1-1 of the raw electronic medical record information DB 144-1 of the existing system.

The conversion unit 126-1 refers to the correspondence relation table 137-1 to acquire data corresponding to each item of the raw medical record information DB 145-1 of the new system from the raw medical record information DB 144-1 of the existing system, and performs a conversion process by writing the acquired data in the raw medical record information DB 145-1 of the new system.

Next, the processing of the concealment unit 124-1 will be described.

The concealment unit 124-1 conceals data of a predetermined item of the raw medical record information DB 145-1 of the new system and writes the concealed data in the concealed medical record DB 154-1.

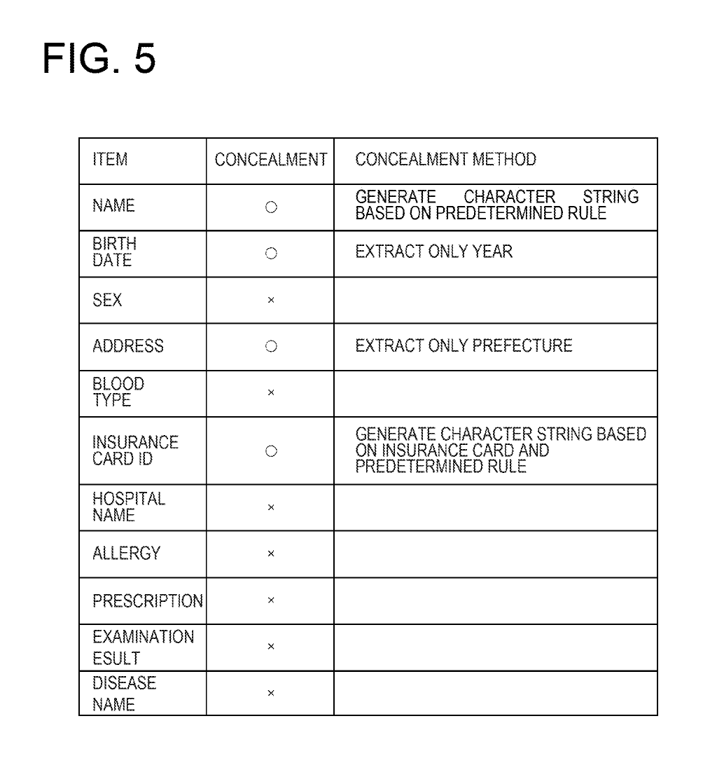

FIG. 5 is a view illustrating items to be concealed and a concealment method. In FIG. 5, items to be concealed are denoted by O and items to be not concealed are denoted by X.

The concealment unit 124-1 conceals a name, a birth date, an address, and an insurance card ID.

The concealment unit 124-1 converts a character string of the name and the insurance card ID based on a predetermined rule, thereby generating a concealed character string of the name and the insurance card ID. The concealed character string is a character string from which the original character string cannot be known by other persons. For example, the concealment unit 124-1 uses a hash function to generate the concealed character string. The concealed character string includes, for example, a number, capital letters and small letters of the alphabet, or a symbol.

The concealment unit 124-1 extracts only the year of the birth date data, thereby generating concealed data of the birth date.

The concealment unit 124-1 extracts only a prefecture of the address, thereby generating a concealed address.

Note that the items to be concealed and the concealment method are only examples and the present disclosure is not limited thereto.

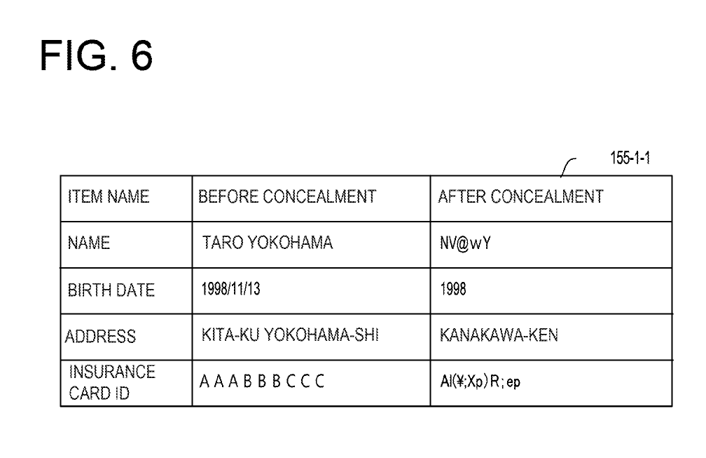

FIG. 6 illustrates an example of a concealment mapping table.

When the concealment unit 124-1 conceals the name=Taro Yokohama, the birth date=1998/11/3, the address=Kita-Ku, Yokohama-shi, Kanagawa-Ken, and the insurance card ID=AAABBBCCC according to the concealment method illustrated in FIG. 5, the concealed name, birth date, address and insurance card ID are NV@wY, 1998, Kanagawa and A|(¥; Xp)R; ep, respectively.

When the concealment unit 124-1 writes the items, the data before concealment and the data after concealment in the concealment mapping table 155-1-1, the concealment mapping table 155-1-1 is as shown in FIG. 6.

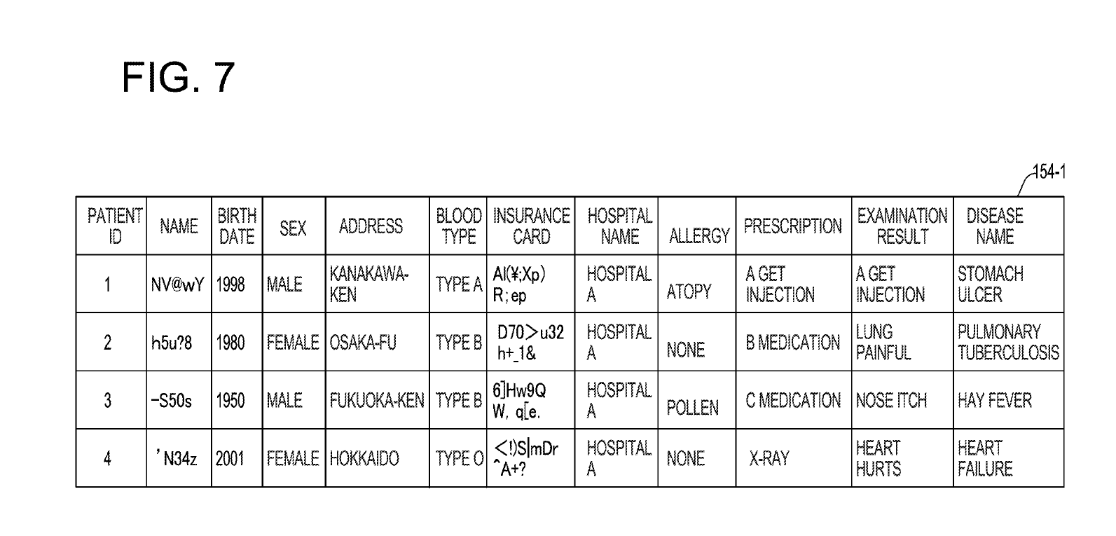

FIG. 7 illustrates an example of a concealment medical record information DB.

The concealment electronic medical record information DB 154-1 includes, as items, a patient ID, a name, a birth date, a sex, an address, a blood type, an insurance card ID, a hospital name, an allergy, a prescription, an examination result and a disease name. The concealment medical record information DB 154-1 has the same items as the raw medical record information DB 145-1 of the new system. That is, the concealment medical record information DB 154-1 includes items of electronic medical record of a format of electronic medical record analyzed by the analysis PC 231 and of the common format common for hospitals. That is, in the concealment medical record information DB 154-1, the concealed electronic medical record is recorded in the common format.

The concealment unit 124-1 writes the data of the raw medical record information DB 145-1 in the concealment medical record information DB 154-1 for the items to be not concealed and write the concealed data in the concealment medical record information DB 154-1 for the items to be concealed. Thus, a new concealment medical record information DB 154-1 is generated as illustrated in FIG. 7.

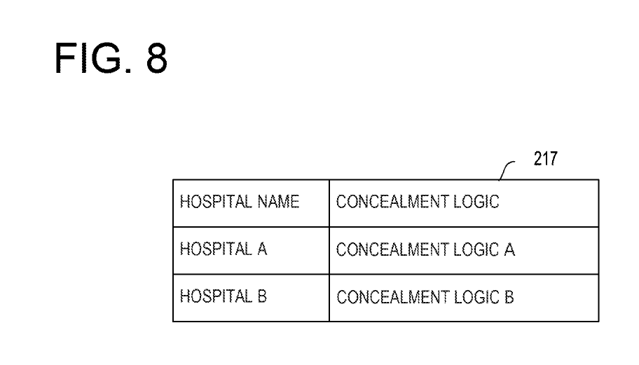

FIG. 8 illustrates an example of a concealment logic distribution management table.

The concealment logic distribution management table 217 includes, as items, a hospital name and a concealment logic, which are recorded in association.

The hospital name is the name of a hospital using the hospital system 101-1 of a concealment logic transmission destination.

The concealment logic is information for identifying concealment logic. In FIG. 8, concealment logic A corresponds to the concealment logic 134-1.

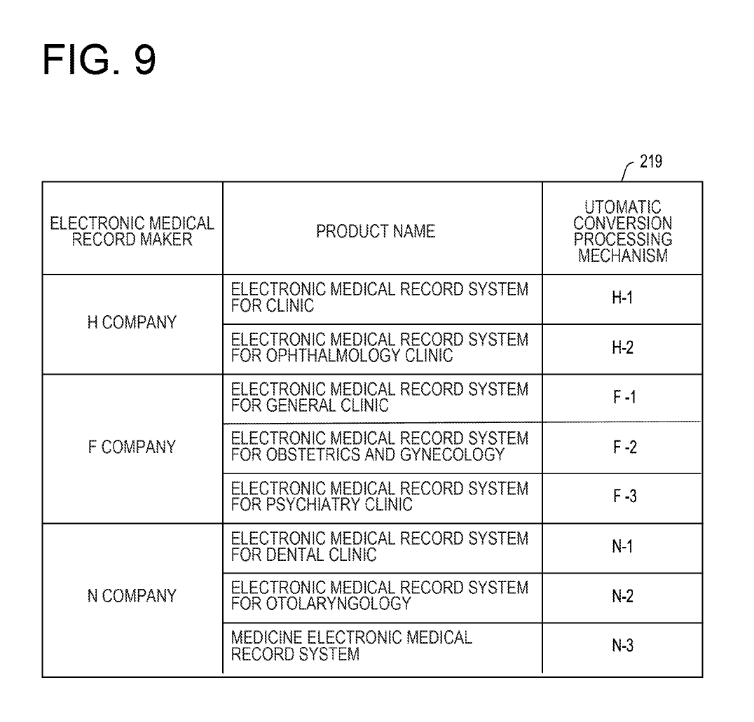

FIG. 9 illustrates an example of an automatic conversion processing mechanism list.

The automatic conversion processing mechanism list 219 includes, as items, an electronic medical record maker, a product name and an automatic conversion processing mechanism, which are recorded in association. When an electronic medical record of the common format is generated by converting an electronic medical record of another format of the raw medical record information DB 144-1 of the existing system, the automatic conversion processing mechanism list 219 indicates which automatic conversion processing mechanism to be appropriately used.

The electronic medical record maker indicates the name of a maker.

The product name indicates the name of a product.

The automatic conversion processing mechanism indicates information for identifying an automatic conversion processing mechanism.

For example, the automatic conversion processing mechanism list 219 of FIG. 9 indicates that the electronic medical record generated by the electronic medical record system for clinic of the H company can be converted into an electronic medical record of the common format by the automatic conversion processing mechanism denoted by H-1 in the automatic conversion processing mechanism group 216.

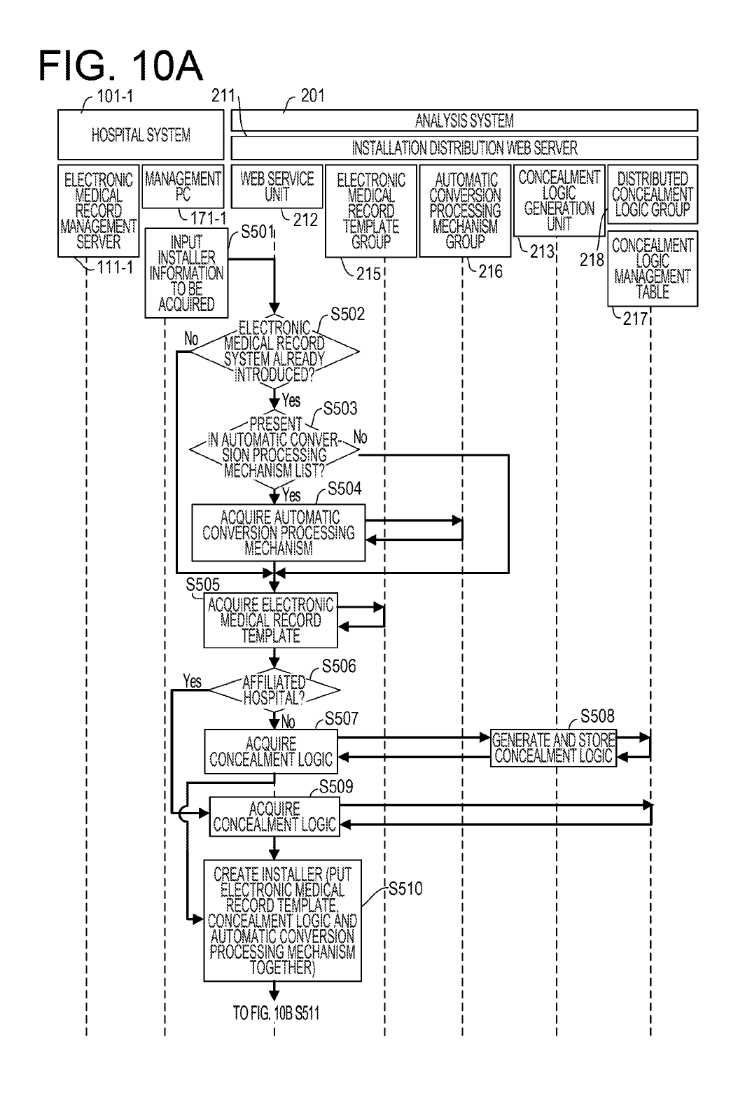

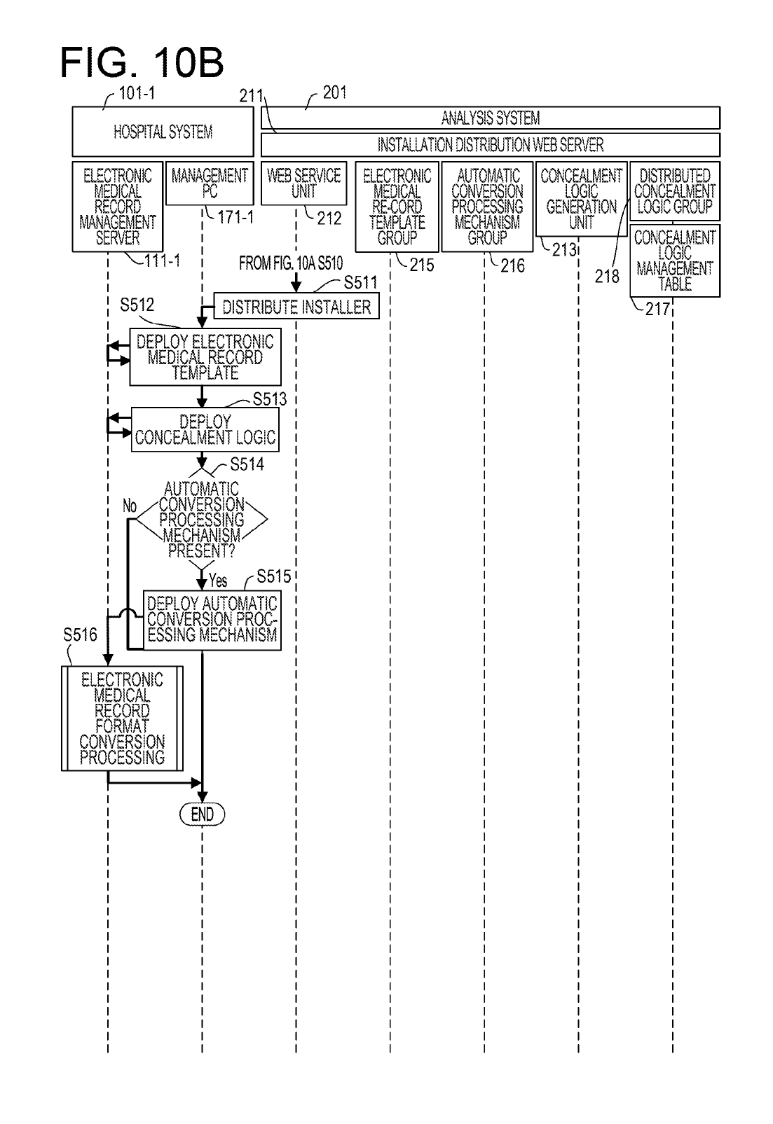

FIGS. 10A and 10B are sequence diagrams of an installation process of an electronic medical record installation set according to an embodiment.

Here, a case where an electronic medical record installation set is installed in the hospital system 101-1 of the hospital A will be described. The same process is also applied to installing an electronic medical record installation set in the hospital system 101-2 of the hospital B.

In step S501, an administrator uses the management PC 171-1 to access the installer distribution web server 211 and input installation information. In addition, the administrator inputs the name of a hospital where the electronic medical record system is installed. Here, since the electronic medical record system is introduced (installed) in the electronic medical record management server 111-1, the administrator inputs the hospital A as the hospital name.

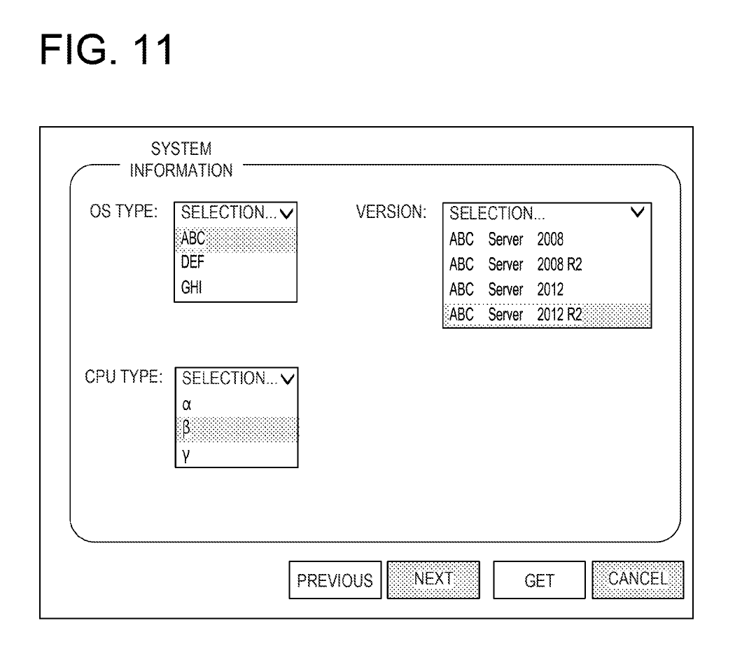

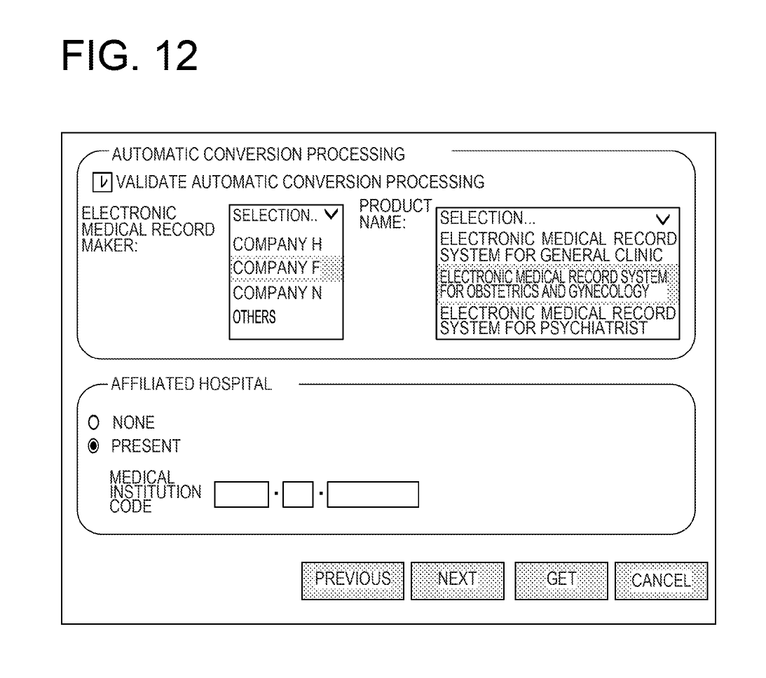

FIGS. 11 and 12 are views illustrating an installation information input screen.

An installation information input screen as illustrated in FIG. 11 is displayed on a display device of the management PC 171-1 and an administrator inputs information of the electronic medical record management server 111-1. The administrator inputs the type and version of an OS and the type of a CPU of the electronic medical record management server 111-1 as installation information.

In addition, an installation information input screen as illustrated in FIG. 12 is displayed on the display device of the management PC 171-1. When performing an automatic conversion process of an electronic medical record of another format included in the raw medical record information DB 144-1 of the existing system into an electronic medical record of the common format, the administrator checks a relevant check box. In addition, as the installation information, a maker name and a product name of an electronic medical record maker used to generate an electronic medical record of another format of the raw medical record information DB 144-1 of the existing system are input. In addition, when there is an affiliation of a hospital where the electronic medical record is installed, the administrator selects a “Yes” radio button of the affiliated hospital on the input screen and inputs a medical institution code indicating the hospital where the electronic medical record is installed.

In step S502, the web service unit 212 determines whether or not an electronic medical record system has already been introduced. When the electronic medical record system has already been introduced, the control proceeds to step S503. When the electronic medical record system has not already been introduced, the control proceeds to step S505. When installation information has been already inputted to perform an automatic conversion process, the web service unit 212 determines that the electronic medical record system has already been introduced.

In step S503, the web service unit 212 determines whether or not the electronic clinical chart maker and the product name of the already introduced electronic medical record system are included in the automatic conversion processing mechanism list 219. When the electronic medical record maker and the product name of the already introduced electronic medical record system are included in the automatic conversion processing mechanism list 219, the control proceeds to step S504. When the electronic medical record maker and the product name of the already introduced electronic medical record system are not included in the automatic conversion processing mechanism list 219, the control proceeds to step S505.

The electronic medical record maker and the product name of the already introduced electronic medical record system has been input in step S501 as the electronic medical record maker name and the product name used to generate the electronic medical record of another format of the raw electronic medical record information DB 144-1 of the existing system.

In step S504, the web service unit 212 refers to the automatic conversion processing mechanism list 219 to acquire an automatic conversion processing mechanism corresponding to the electronic medical record maker and the product name of the already introduced electronic medical record system from the automatic conversion processing mechanism group 216.

In step S505, the web service unit 212 acquires an electronic medical record template corresponding to the installation information from the electronic medical record template group 215.

In step S506, the web service unit 212 determines whether or not a concealment logic has already been distributed to an affiliation of a hospital where the electronic medical record system is introduced. When the concealment logic has already been distributed to the affiliated hospital, the control proceeds to step S509. When the concealment logic has not already been distributed to the affiliated hospital, the control proceeds to step S507. When the “Yes” radio button of the affiliated hospital on the input screen is selected in the input of installation information in step S501, the web service unit 212 determines that the concealment logic has been distributed.

In step S507, the web service unit 212 requests the concealment logic generation unit 213 to generate a concealment logic.

In step S508, the concealment logic generation unit 213 generates the concealment logic. The web service unit 212 acquires the generated concealment logic from the concealment logic generation unit 217. The concealment logic generation unit 213 includes the generated concealment logic in the distributed concealment logic group 218 and writes information indicating the generated concealment logic and the name of a hospital to which the generated concealment logic is distributed (that is, the name of a hospital in which the electronic medical record system is introduced) in the concealment logic management table 217. The concealment logic generation unit 213 may generate concealment logics having different items to be concealed and different concealment methods for different hospitals in which the electronic medical record system is introduced. In addition, when an item to be concealed is preset for each hospital, a concealment logic corresponding to the setting may be generated.

In step S509, the web service unit 212 refers to the concealment logic management table 217 to acquire a concealment logic distributed to the affiliated hospital among the concealment logics included in the distributed concealment logic group 218. For example, in a case where an introduction destination of the electronic medical record is a hospital X and a medical institution code of the hospital X is inputted in the input of the installation information, the web service unit 212 acquires the hospital name of an affiliation of the hospital X based on the input medical institution code. The storage unit 214 stores information indicating the hospital name and the affiliated hospital corresponding to the medical institution code and the web service unit 212 refers to the stored information to acquire the hospital name of an affiliation of a hospital. Then, the web service unit 212 detects a concealment logic corresponding to the acquired hospital name of the affiliated hospital from the concealment logic management table 217 and acquires a concealment logic corresponding to the detected concealment logic from the distributed concealment logic group 218. For example, when the affiliation of the hospital X is a hospital B, by referring to the concealment logic table 217 of FIG. 8, a concealment logic B corresponding to the hospital B is detected and is acquired from the distributed concealment logic group 218. In addition, the web service unit 212 writes information indicating the acquired concealment logic and the name of the hospital in which the electronic medical record is introduced, in the concealment logic management table 217. By using the same concealment logic at affiliated hospitals, it is possible to maintain compatibility of electronic medical record among the affiliated hospitals. When the concealment logic corresponding to the hospital name of the affiliated hospital obtained by the web service unit 212 cannot be detected from the concealment logic table 217, the control proceeds to step S507.

In step S510, the web service unit 212 creates an electronic medical record installation set. The electronic medical record installation set includes the acquired electronic medical record template, generated concealment logic and acquired automatic conversion processing mechanism.

In step S511, the web service unit 212 transmits the electronic medical record installation set to the management PC 171. The management PC 171 stores the electronic medical record installation set in the storage unit 131-1 of the electronic medical record management server 111-1.

In step S512, the management PC 171 installs the electronic medical record template 133-1 in the electronic medical record management server 111-1, and the electronic medical record management server 111-1 executes the electronic medical record template 133-1. As a result, the electronic medical record template 133-1 operates as the reading unit 122-1, the writing unit 123-1, the restoration unit 125-1 and the data transmission unit 127-1.

In step S513, the management PC 171 installs the concealment logic 134-1 in the electronic medical record management server 111-1, and the electronic medical record management server 111-1 executes the concealment logic 134-1. As a result, the concealment logic 134-1 operates as the concealment unit 124-1.

In step S514, when the automatic conversion processing mechanism 135-1 is included in the electronic medical record installation set 132-1, the control proceeds to step S515. When the automatic conversion processing mechanism 135-1 is not included, the process is ended.

In step S515, the management PC 171 installs the conversion processing program 136-1 in the electronic medical record management server 111-1, and the electronic medical record management server 111-1 executes the conversion processing program 136-1. As a result, the conversion processing program 136-1 operates as the conversion unit 126-1.

In step S516, the electronic medical record management server 111-1 performs an electronic medical record format conversion process. By the electronic medical record format conversion process, an electronic medical record of another format of the raw medical record information DB 144-1 of the existing system is converted into an electronic medical record of the common format which is then stored in the raw medical record information DB 145-1 of the new system.

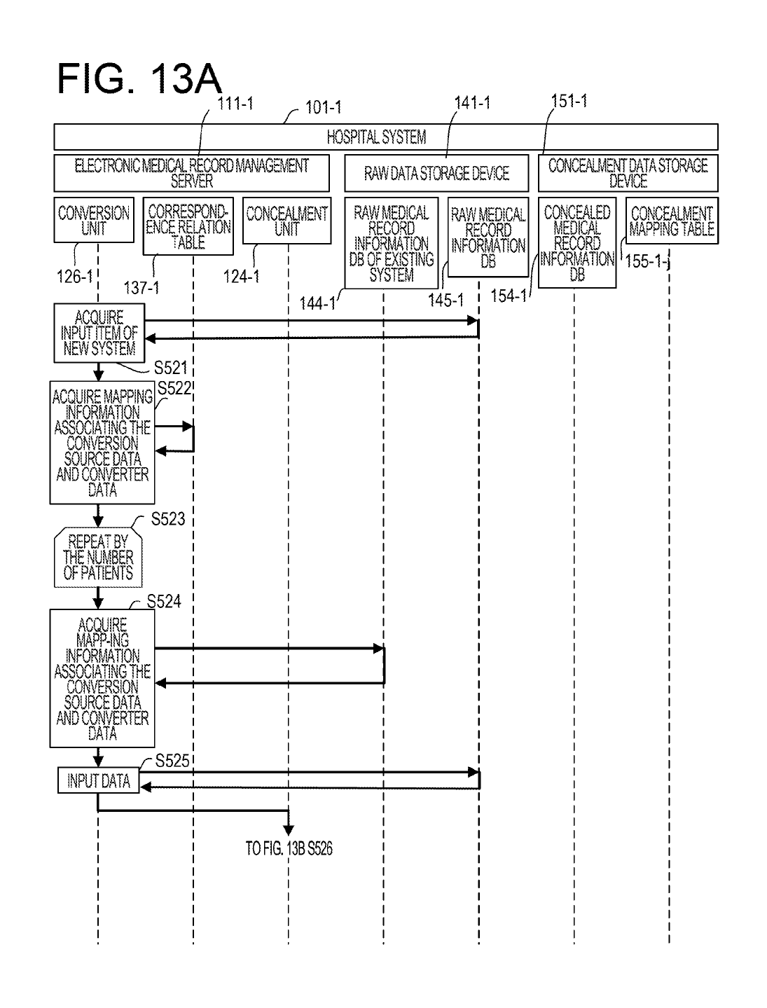

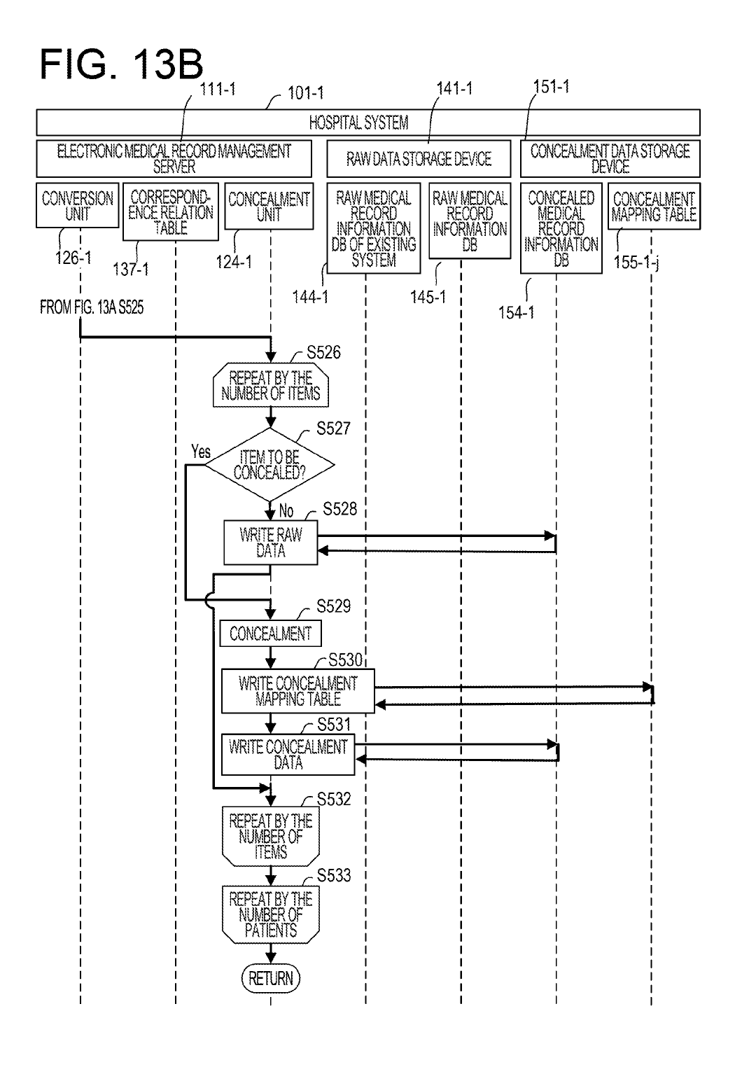

FIGS. 13A and 13B are detailed sequence diagrams of the electronic medical record format conversion process.

In step S521, the converting unit 126-1 acquires an item of the raw medical record information DB 145-1 of the new system (that is, an item of the electronic medical record of the common format) from the raw electronic medical record information DB 145-1 of the new system.

In step S522, the conversion unit 126-1 refers to the correspondence relation table 137-1 to acquire the correspondence (mapping information) between the conversion source data and the converted data. That is, information indicating a position of the raw medical record information DB 144-1 of the existing system at which the data of each item of the raw medical record information DB 145-1 of the new system exists is acquired.

Step S523 is the beginning of a loop corresponding to step S533, in which one unselected patient ID among the patient IDs included in the raw medical record information DB 144-1 of the existing system is selected. Hereinafter, the selected patient ID is referred to as a selection patient ID. A loop between step S523 and step S533 is repeated by the number of patients (the number of patient IDs) included in the raw medical record information DB 144-1 of the existing system. For example, in the raw medical record information DB 144-1 of the existing system illustrated in FIG. 2, the maximum value of the patient ID is 4 and the electronic medical record of 4 patients are included.

In step S524, the conversion unit 126-1 acquires data corresponding to each item of the raw medical record information DB 145-1 of the new system corresponding to the selection patient ID from the raw medical record information DB 144-1 of the existing system. For example, for the selection patient ID=1, the conversion unit 126-1 converts the data of each item corresponding to the patient ID=1 of the raw medical record information DB 145-1 of the new system from the data corresponding to the patient ID=1 of the raw medical record information DB 144-1 of the existing system.

In step S525, the conversion unit 126-1 writes the selection patient ID and the acquired data corresponding to each item of the raw medical record information DB 145-1 of the new system in the raw medical record information DB 145-1 of the new system.

Step S526 is the beginning of a loop corresponding to step S532 and one unselected item among the items of the raw medical record information DB 145-1 of the new system is selected. Hereinafter, the selected item is referred to as a selection item.

In step S527, the concealment unit 124-1 determines whether or not the selection item is an item to be concealed. When the selection item is an item to be concealed, the control proceeds to step S529. When the selection item is not an item to be concealed, the control proceeds to step S528. The item to be concealed is preset.

In step S528, the concealment unit 124-1 writes the data of the selection item of the raw medical record information DB 145-1 in the concealed medical record information DB 154-1.

In step S529, the concealment unit 124-1 conceals the data of the selection item of the raw medical record information DB 145-1.

In step S530, the concealment unit 124-1 writes the data of the selection item of the raw medical record information DB 145-1 and the concealed data of the selection item in the concealment mapping table 155-1-j in association. When there is no concealment mapping table 155-1-j corresponding to the selection patient ID in the storage unit 153-1, the concealment unit 124-1 creates a concealment mapping table 155-1-j prior to writing.

In step S531, the concealment unit 124-1 writes the concealed data of the selection item in the concealed medical record information DB 154-1.

Step S532 is the end of the loop corresponding to step S526. When all the items of the raw medical record information DB 145-1 of the new system have been selected, the control proceeds to step S533. When all the items of the raw medical record information DB 145-1 of the new system have not been selected, the control returns to step S526.

Step S533 is the end of the loop corresponding to step S523. When all the patient IDs of the raw medical record information DB 144-1-1 of the existing system have been selected, the electronic medical record format conversion process is ended. When all the patient IDs of the raw medical record information DB 144-1-1 of the existing system have not been selected, the control returns to step S523.

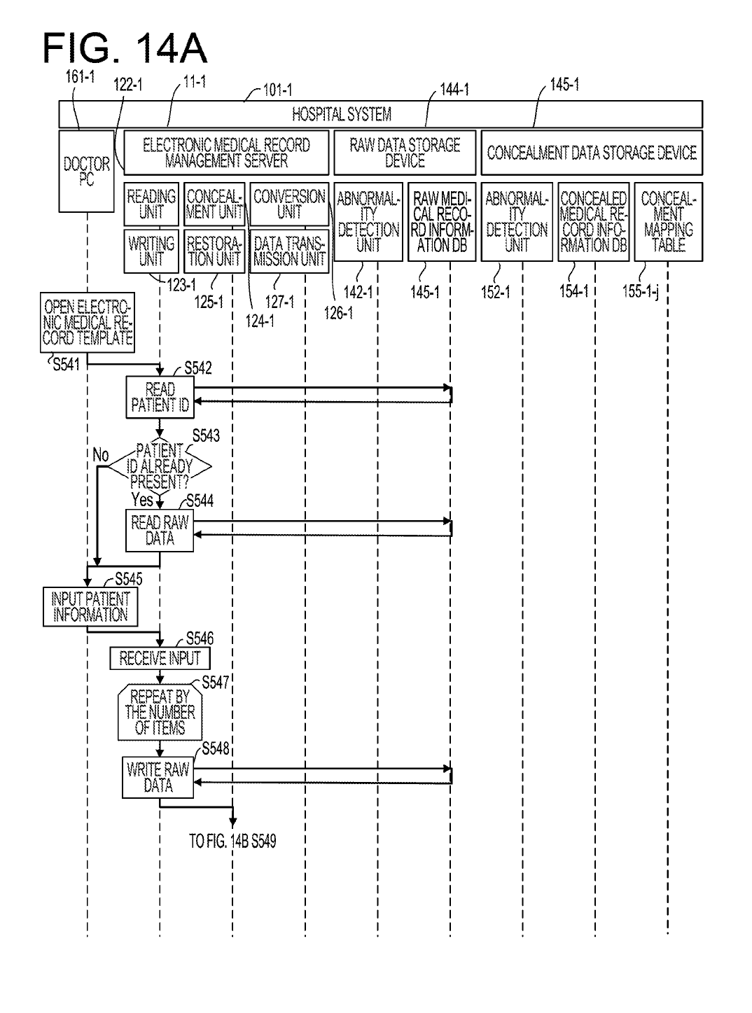

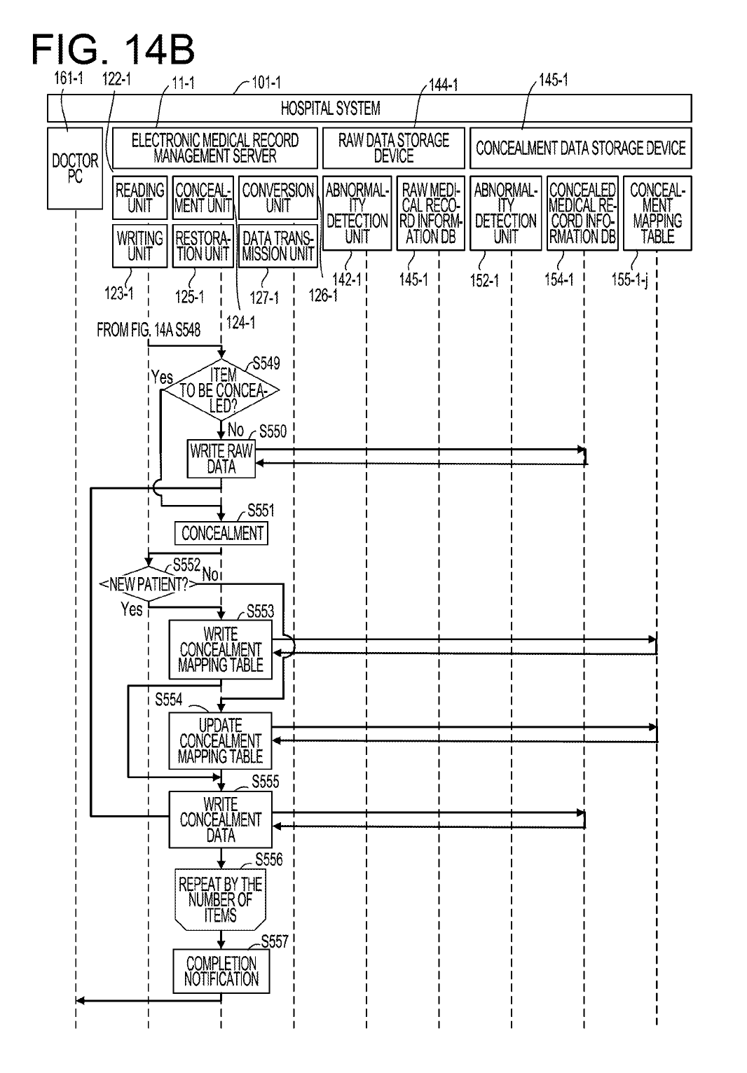

FIGS. 14A and 14B are sequence diagrams of an electronic medical record input process according to an embodiment.

It is assumed that the installation process of FIGS. 10A and 10B has already been performed.

In step S541, a doctor uses the doctor PC 161 to access the electronic medical record management server 111-1 and input an electronic medical record. The doctor inputs a patient ID and the doctor PC 161 transmits the patient ID to the electronic medical record management server 111-1.

In step S542, the reading unit 122-1 reads the patient ID of the raw medical record information DB 145-1 of the new system.

In step S543, the reading unit 122-1 determines whether or not the patient ID input from the doctor PC 161 exists in the raw medical record information DB 145-1. When the patient ID input from the doctor PC 161 exists in the raw medical record information DB 145-1, the control proceeds to step S544. When the patient ID input from the doctor PC 161 does not exist in the raw medical record information DB 145-1, the control proceeds to step S545.

In step S544, the reading unit 122-1 reads an electronic medical record corresponding to the inputted patient ID from the raw medical record information DB 145-1 and displays it on the display device of the doctor PC 161-1.

In step S545, the doctor uses the doctor PC 161 to input data of a patient to an electronic medical record using the common format. When the patient is a new patient, the doctor inputs data of all the items of the electronic medical record. When the patient is not a new patient, the doctor inputs data of an item requiring updating among plural items of the electronic medical chart. The doctor PC 161 transmits the input item data to the writing unit 123-1.

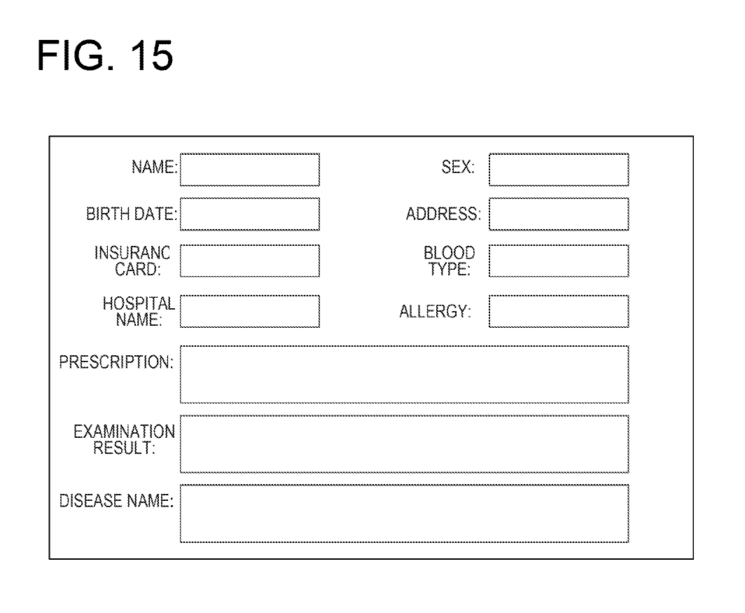

FIG. 15 illustrates an example of an input screen of an electronic medical record using the common format displayed on the display device of the doctor PC 161. The input screen of FIG. 15 includes items of a name, a sex, a birth date, an address, an insurance card ID, a blood type, a hospital name, an allergy, a prescription, an examination result and a disease name of a patient.

In step S546, the writing unit 123-1 receives the item data input from the doctor PC 161.

Step S547 is the beginning of the loop corresponding to step S556, in which one unselected item among the input items is selected. Hereinafter, the selected item is referred to as a selection item.

In step S548, the writing unit 123-1 writes the data of the selection item among the data of the input items received from the doctor PC 161 in the raw medical record information DB 145-1.

In step S549, the reading unit 122-1 determines whether or not the selection item is an item to be concealed. When the selection item is an item to be concealed, the control proceeds to step S551. When the selection item is not an item to be concealed, the control proceeds to step S550. The item to be concealed is preset.

In step S550, the concealment unit 124-1 writes the data of the selected item in the concealed medical record information DB 154-1.

In step S551, the concealment unit 124-1 conceals the data of the selection item of the raw medical record information DB 145-1.

In step S552, the reading unit 122-1 determines whether or not the patient ID input from the doctor PC 161 is a new patient ID. When the input patient ID is a new patient ID, the control proceeds to step S553. When the input patient ID is not a new patient ID, the control proceeds to step S554.

In step S553, the concealment unit 124-1 writes the data of the selection items of the raw medical record information DB 145-1 and the concealed data of the selection item in the concealment mapping table 155-1-j corresponding to the input patient ID in association. When there is no concealment mapping table 155-1-j corresponding to the inputted patient ID in the storage unit 153-1, the concealment unit 124-1 creates a concealment mapping table 155-1-j corresponding to the input patient ID.

In step S554, the concealment unit 124-1 updates the concealment mapping table 155-1-j corresponding to the patient ID input in association of the data of the selection item of the raw medical record information DB 145-1 with the concealed data of the selection item.

In step S555, the concealment unit 124-1 writes the concealed data of the selection item in the concealed medical record information DB 154-1.

Step S556 is the end of the loop corresponding to step S547. When all the input items have been selected, the control proceeds to the step S557. When all the input items have not been selected, the control returns to step S547.

In step S557, the concealment unit 124-1 transmits an electronic medical record write completion notification to the doctor PC 161-1.

Next, a process of transmitting the concealed data (that is, the concealed electronic medical record) to the analysis system 201 will be described.

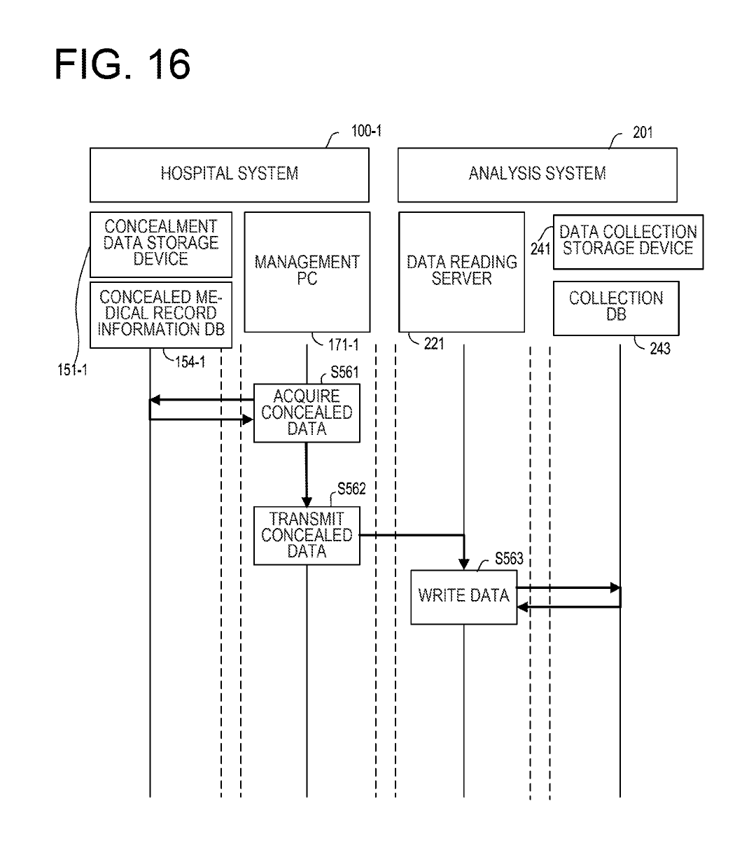

FIG. 16 is a sequence diagram of a data transfer process according to an embodiment.

In step S561, the management PC 171-1 acquires the concealed medical record information DB 154-1 including the concealment electronic medical record of the common format. More specifically, the management PC 171-1 requests the electronic medical record management server 111-1 for the concealed medical record information DB 154-1. Upon receiving the request, the electronic medical record management server 111-1 reads the concealed medical record information DB 154-1 from the concealment data storage device 151-1 and transmits it to the management PC 171-1. In addition, the electronic medical record management server 111-1 may transmit the concealed medical record information DB 154-1 to the data reading server 221.

In step S562, the management PC 171-1 transmits the concealed medical record information DB 154-1 to the data reading server 221.

In step S563, the data reading server 221 writes the concealment electronic medical record of the common format included in the concealed medical record information DB 154-1 in the collection DB 243.

Next, a process of analyzing data included in the collection DB 243 will be described.

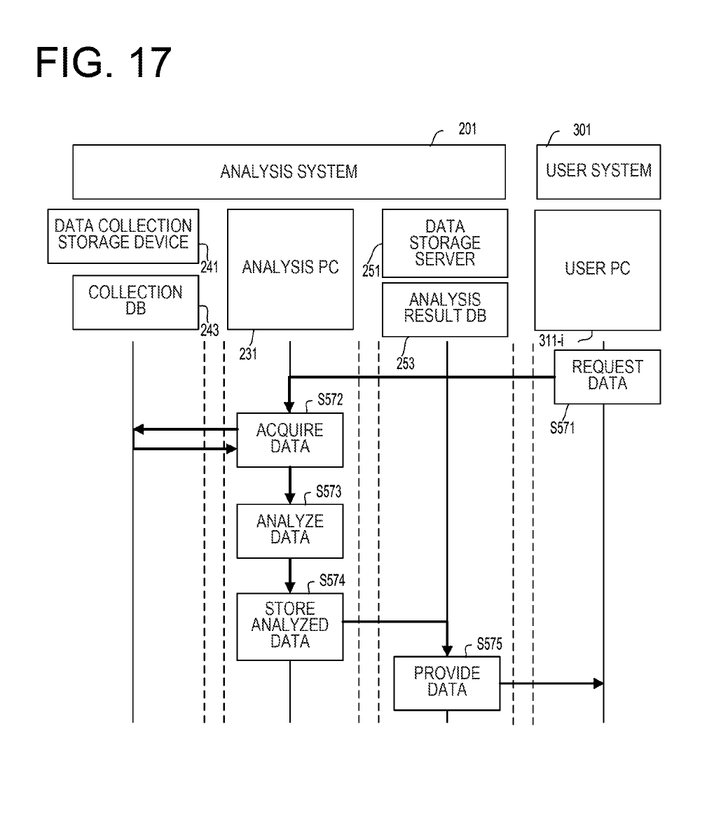

FIG. 17 is a sequence diagram of a data analysis process according to an embodiment.

In step S571, the user PC 311-i requests the analysis PC 231 for an analysis result.

In step S572, the analysis PC 231 acquires the collection DB 243 including the concealment electronic medical record of the common format from the data collection storage device 241.

In step S573, the analysis PC 231 analyzes the concealment electronic medical record contained in the collection DB 243.

In step S574, the analysis PC 231 writes the analysis result in the analysis result DB 253.

In step S575, the data storage server 251 transmits the analysis result included in the analysis result DB 253 to the user PC 311-i.

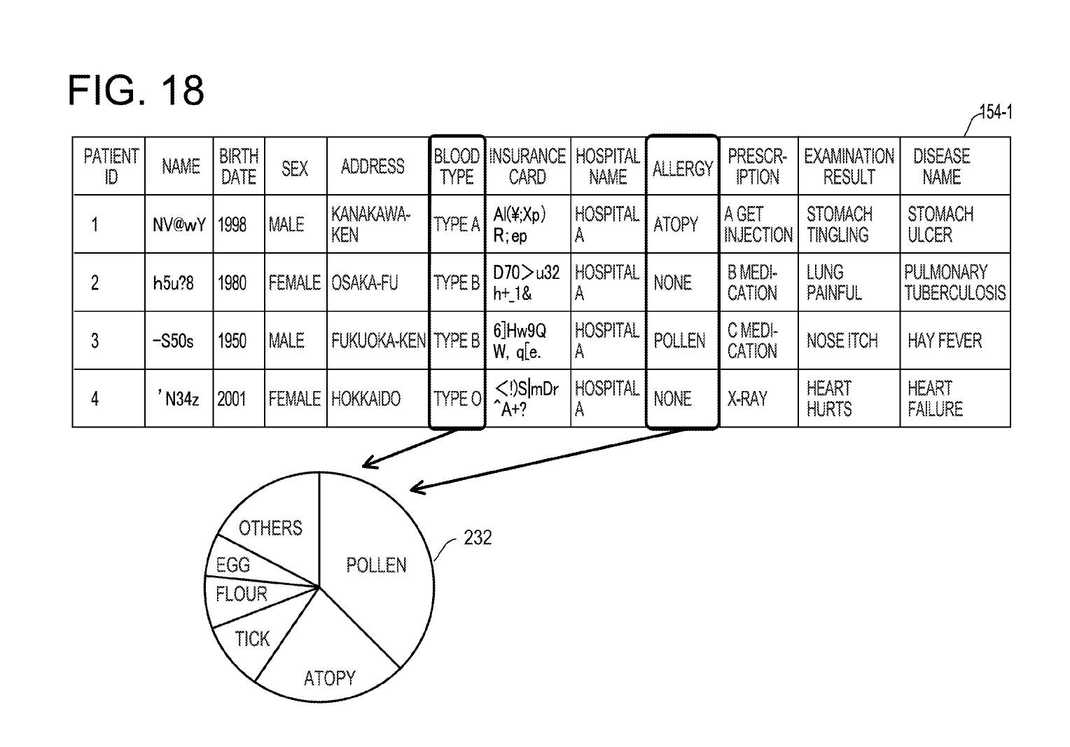

FIG. 18 is a view illustrating an example of an analysis process of the concealment electronic medical record.

FIG. 18 illustrates an example of an analysis process for a concealment electronic medical record acquired from the hospital system 101-1. In the analysis process of FIG. 18, an analysis pattern including items of blood type and allergy is used in order to extract an allergy that a patient with the blood type B has. As a result, “none” is extracted as the allergy of a patient of an ID “2” having the blood type B and “pollen” is extracted as the allergy of a patient of an ID “3” having the blood type B.

In addition to the patients with ID “1” to ID “4”, when the entries of other patients having an allergy are included in the collection DB 243, a pie chart 232 representing the proportion of each of plural allergies among all entries of the patient having the blood type B is generated. Then, the analysis result including the pie chart 232 is stored in the analysis result DB 253. From the pie chart 232, it can be seen that the patients with the blood type B have the allergy of “pollen” and “atopy”.

An example of the analysis process for the concealment electronic medical record acquired from the hospital system 101-1 has been illustrated in FIG. 18. However, an analysis process may be performed for concealment electronic medical record acquired from a plurality of hospital systems 101-i, further including a concealment electronic medical record acquired from the hospital system 101-2.

As described above, in each hospital system 101-i, since an electronic medical record is created in or converted into the common format common for various hospitals, the analysis PC 231 does not need to perform a process for aligning the formats of electronic medical record, thereby shortening the processing time.

Next, a restoration ¥process in a case where raw data (that is, an electronic medical record not concealed) is broken will be described.

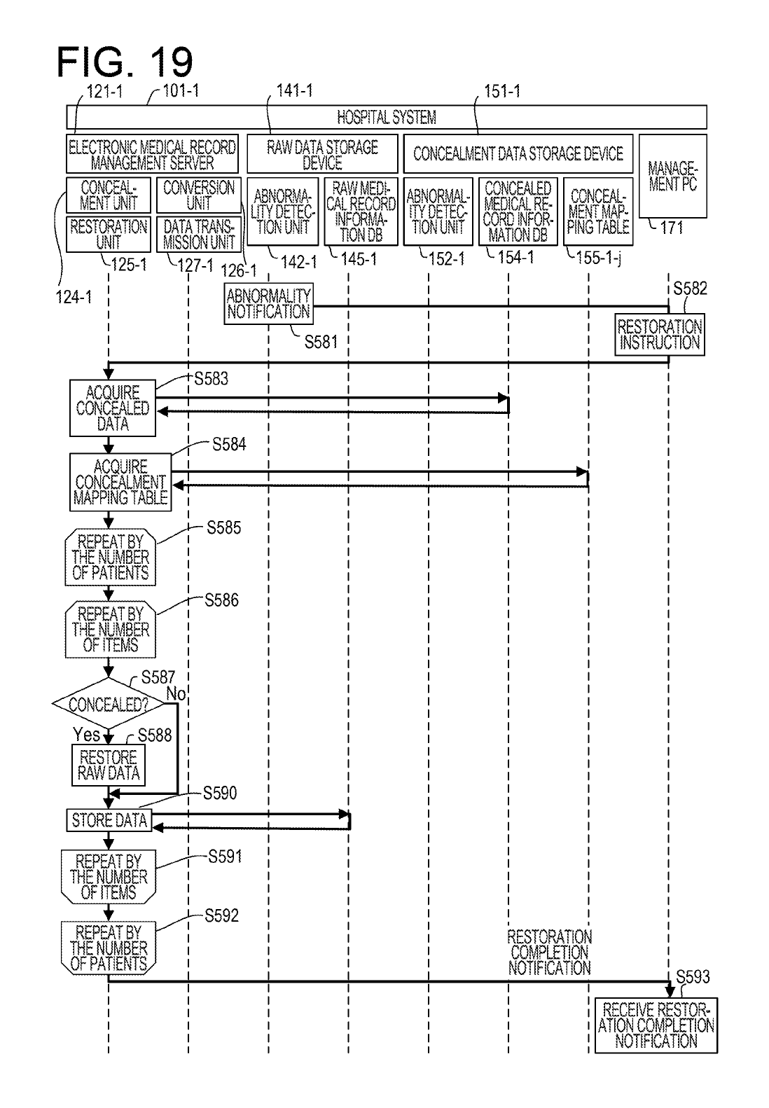

FIG. 19 is a sequence diagram of a raw electronic medical record restoration process according to an embodiment.

It is here assumed that the raw medical record information DB 144-1, the concealed medical record information DB 154-1 and the concealment mapping table 155-1-j have been already generated by the installation process of FIGS. 10A and 10B and the electronic medical record input process of FIGS. 14A and 14B.

In step S581, the abnormality detection unit 142-1 of the raw data storage apparatus 141-1 detects an abnormality of the raw electronic medical record information DB 145-1. That is, the abnormality detection unit 142-1 detects the effect that the data in the raw medical record information DB 145-1 is damaged and cannot be read. The abnormality detection unit 142-1 notifies the management PC 171 of the abnormality.

In step S582, the management PC 171 transmits a restoration instruction to the restoration unit 125-1.

In step S583, the restoration unit 125-1 acquires the concealed electronic medical record information DB 154-1 from the concealment data storage device 151-1.

In step S584, the restoration unit 125-1 acquires the concealment mapping table 155-1-j from the concealment data storage device 151-1.

Step S585 is the beginning of the loop corresponding to step S592, in which one unselected patient ID among the patient IDs included in the concealed electronic medical record information DB 154-1 is selected. Hereinafter, the selected patient ID is referred to as a selection patient ID.

Step S586 is the beginning of the loop corresponding to step S591, in which one unselected item among the items of the concealed electronic medical record information DB 154-1 is selected. Hereinafter, the selected item is referred to as a selection item.

In step S587, the restoration unit 125-1 determines whether or not data of the selection item is concealed. When the data of the selected item is concealed, the control proceeds to step S588. When it is not concealed, the control proceeds to step S590. When the selection item is recorded in the concealment mapping table 155-1-j, the restoration unit 125-1 determines that the data of the selection item has been concealed.

In step S588, the restoration unit 125-1 acquires (restores) the data before concealment corresponding to the concealed data of the selection item of the selection patient ID based on the concealment mapping table 155-1-j. For example, “Taro Yokohama” before concealment corresponding to “NV@wY” after concealing the “name” of the concealment mapping table 155-1-1 of FIG. 6 corresponding to the selection patient ID=1 is acquired as data of “name” before concealment.

In step S590, the restoration unit 125-1 writes the data before concealment of the selection item of the selection patient ID in the raw electronic medical record information DB 145-1. Specifically, when the data of the selection item is concealed, the restoration unit 125-1 writes the data before concealment acquired in step S588 in the raw medical record information DB 145-1. When the data of the selection item is not concealed, the restoration unit 125-1 writes the data of the selection item of the selection patient ID in the concealed medical record information DB 154-1 in the raw medical record information DB 145-1.

Step S591 is the end of the loop corresponding to step S586. When all the items of the concealed electronic medical record information DB 154-1 have been selected, the control proceeds to step S592. When all the items of the concealed chart record information DB 154-1 have not been selected, the control returns to step S586.

Step S592 is the end of the loop corresponding to step S585. When all the patient IDs of the concealed medical record information DB 154-1 have been selected, the control proceeds to step S592. When all the records of the concealed medical record information DB 154-1 have not been selected, the control returns to step S585.

In step S593, the management PC 171 receives a restoration completion notification.

Next, a restoration process in a case where concealment data (that is, a concealed electronic medical record) or the concealment mapping table 155-1-j is broken will be described.

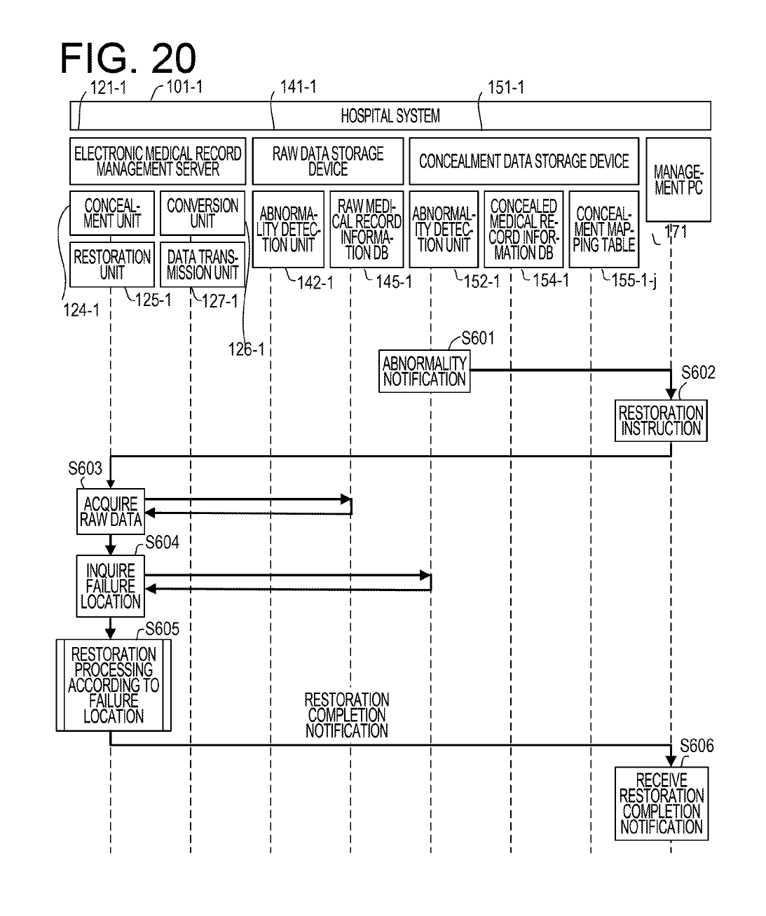

FIG. 20 is a sequence diagram of a restoration process of a concealment electronic medical record and a concealment mapping table according to an embodiment.

It is here assumed that the raw medical record information DB 144-1, the concealed electronic medical record information DB 154-1 and the concealment mapping table 155-1-j have been already generated by the installation process of FIGS. 10A and 10B and the electronic medical record input process of FIGS. 14A and 14B.

In step S601, the abnormality detection unit 152-1 of the concealment data storage apparatus 151-1 detects an abnormality of at least one of the concealed medical record information DB 154-1 and the concealment mapping table 155-1-j. That is, the abnormality detection unit 152-1 detects the effect that that the data of at least one of the concealed medical record information DB 154-1 and the concealment mapping table 155-1-j is damaged and cannot be read. The abnormality detection unit 152-1 notifies the management PC 171 of the abnormality.

In step S602, a restoration instruction is transmitted to the restoration unit 125-1.

In step S603, the restoration unit 125-1 acquires the raw medical record information DB 145-1 from the raw data storage device 141-1.

In step S604, the restoration unit 125-1 inquires the abnormality detection unit 152-1 of a failure location (that is, which data is damaged and cannot be read). The abnormality detection unit 152-1 notifies the restoration unit 125-1 of the failure location. More specifically, the abnormality detection unit 152-1 notifies one of (1) the fact that only the concealed medical record DB 154-1 has failed (cannot be read), (2) the fact that only the concealment mapping table 155-1-j has failed (cannot be read out) and (3) the fact that both of the concealed medical record information DB 154-1 and the concealment mapping table 155-1-j have failed (cannot be read).

In step S605, the concealment unit 124-1 and the restoration unit 125-1 perform the restoration process according to the notified failure location. After completion of the restoration process, the restoration unit 125-1 transmits a restoration completion notification to the management PC 171. The details of the restoration process according to the failure location will be described later.

In step S606, the management PC 171 receives the restoration completion notification.

Next, the details of the restoration process according to the failure location will be described.

The concealment unit 124-1 and the restoration unit 125-1 perform a restoration process for one of the following cases (1) to (3) according to the failure location.

(1) A Case Where Only the Concealed Medical Record Information DB 154-1 has Failed

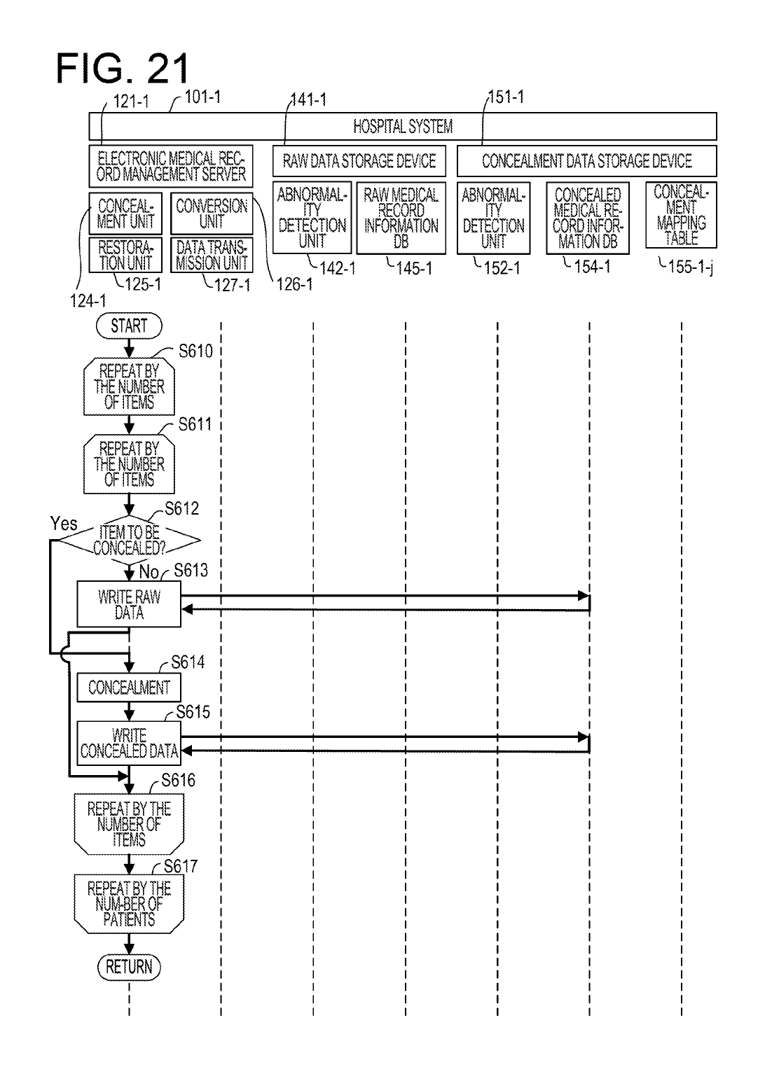

FIG. 21 is a detailed sequence diagram of a restoration process in a case where only the concealed medical record information DB has failed (that is, a case where only the concealed medical record information DB cannot be read).

Step S610 is the beginning of the loop corresponding to step S617, in which one unselected patient ID among the patient IDs included in the raw medical record information DB 145-1 of the new system. Hereinafter, the selected patient ID is referred to as a selection patient ID.

Step S611 is the beginning of the loop corresponding to step S616, in which one unselected item among the items of the raw medical record information DB 145-1 is selected. Hereinafter, the selected item is referred to as a selection item.

In step S612, the restoration unit 125-1 determines whether or not the selection item is an item to be concealed. When the selection item is an item to be concealed, the control proceeds to step S614. When the selection item is not an item to be concealed, the control proceeds to step S613. The item to be concealed is preset.

In step S613, the restoration unit 125-1 writes the data of the selection item of the selection patient ID of the raw medical record information DB 145-1 in the concealed medical record information DB 154-1.

In step S614, the concealment unit 124-1 conceals the data of the selection item of the selection patient ID of the raw medical record information DB 145-1.

In step S615, the restoration unit 125-1 writes the concealed data of the selection item of the selection patient ID in the concealed medical record information DB 154-1.

Step S616 is the end of the loop corresponding to step S611. When all the items of the raw medical record information DB 145-1 have been selected, the control proceeds to step S617. When all the items of the raw electronic medical record information DB 145-1 have not been selected, the control returns to step S611.

The step S617 is the end of the loop corresponding to step S610. When all the patient IDs of the raw electronic medical record information DB 145-1 of the new system have been selected, the restoration process is ended. When all the patient IDs of the raw electronic medical record information DB 145-1 of the new system have not been selected, the control returns to step S610.

(2) Case Where Only the Concealment Mapping Table 155-1-j has Failed

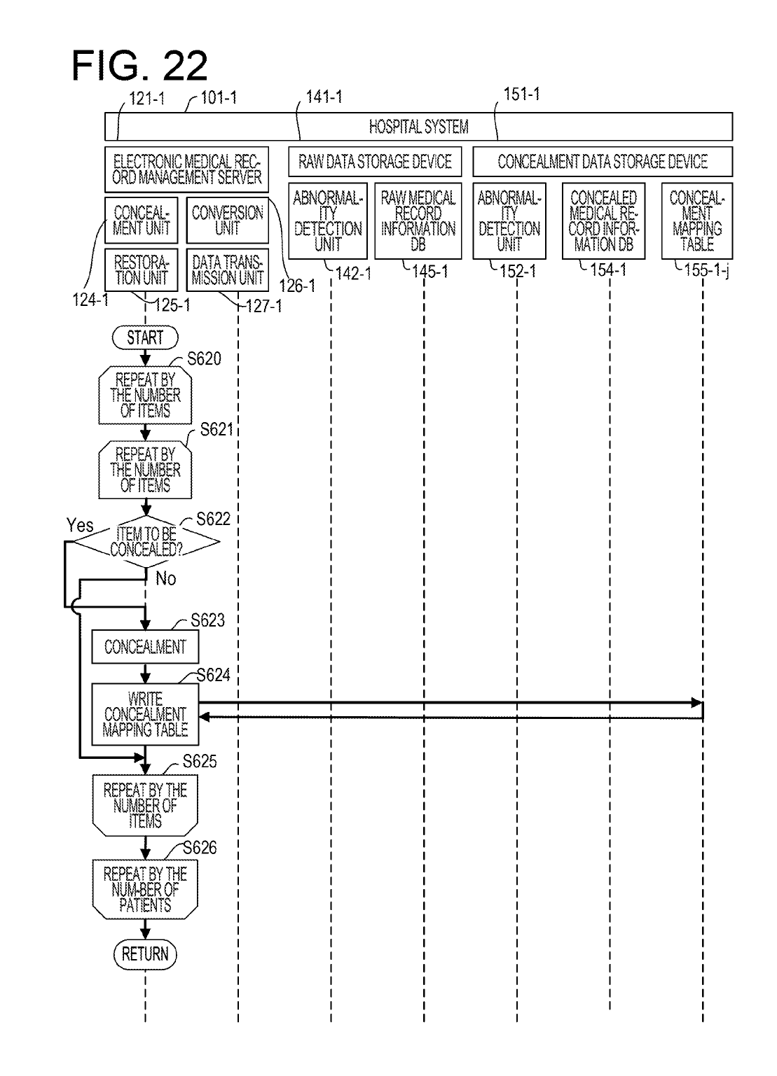

FIG. 22 is a detailed sequence diagram of a restoration process in a case where only the concealment mapping table has failed (that is, in a case where only the concealment mapping table cannot be read).

Step S620 is the beginning of the loop corresponding to step S626, in which one unselected patient ID among the patient IDs included in the raw medical record information DB 145-1 of the new system is selected. Hereinafter, the selected patient ID is referred to as a selection patient ID.

Step S621 is the beginning of the loop corresponding to step S625, in which one unselected item among the items of the raw medical record information DB 145-1 is selected. Hereinafter, the selected item is referred to as a selection item.

In step S622, the restoration unit 125-1 determines whether or not the selection item is an item to be concealed. When the selection item is an item to be concealed, the control proceeds to step S623. When the selection item is not an item to be concealed, the control proceeds to step S625. The Item to be concealed is preset.

In step S623, the concealment unit 124-1 conceals data of the selection item of the selection patient ID of the raw medical record information DB 145-1.

In step S624, the restoration unit 125-1 writes the data of the selection item of the selection patient ID of the raw medical record information DB 145-1 and the concealed data of the selection item in the concealment mapping table 155-1-j corresponding to the selection patient ID in association.

Step S625 is the end of the loop corresponding to step S621. When all the items of the raw medical record information DB 145-1 have been selected, the restoration process is ended. When all the items of the raw medical record information DB 145-1 have not been selected, the control returns to step S621.

Step S626 is the end of the loop corresponding to step S620. When all the patient IDs of the raw medical record information DB 145-1 of the new system have been selected, the restoration process is ended. When all the patient IDs of the raw medical record information DB 145-1 of the new system have not been selected, the control returns to step S520.

(3) Case where both of the concealed electronic medical record information DB 154-1 and the concealment mapping table 155-1-j have failed

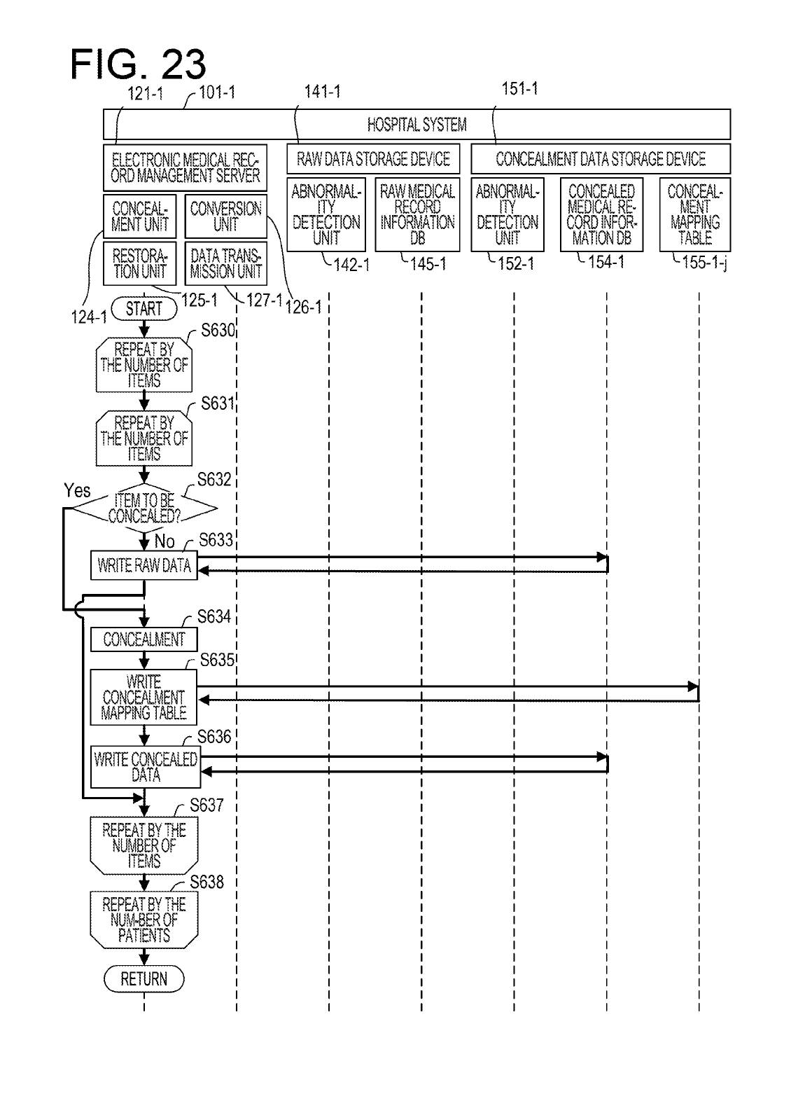

FIG. 23 is a detailed sequence diagram of a restoration process in a case where both of the concealed medical record information DB and the concealment mapping table have failed (that is, in a case where both of the concealed medical record information DB and the concealment mapping table cannot be read).

Step S630 is the beginning of the loop corresponding to step S638, in which one unselected patient ID among the patient IDs included in the raw medical record information DB 145-1 of the new system is selected. Hereinafter, the selected patient ID is referred to as a selection patient ID.

Step S631 is the beginning of the loop corresponding to step S637, in which one unselected item among the items of the raw medical record information DB 145-1 is selected. Hereinafter, the selected item is referred to as a selection item.

In step S632, the restoration unit 125-1 determines whether or the selection item is an item to be concealed. When the selection item is an item to be concealed, the control proceeds to step S634. When the selection item is not an item to be concealed, the control proceeds to step S633. The item to be concealed is preset.

In step S633, the restoration unit 125-1 writes the data of the selection item of the selection patient ID of the raw medical record information DB 145-1 in the concealed medical record information DB 154-1.

In step S634, the concealment unit 124-1 conceals the data of the selection item of the selection patient ID of the raw medical record information DB 145-1.

In step S635, the restoration unit 125-1 writes the data of the selection item of the selection patient ID of the raw medical record information DB 145-1 and the concealed data of the selection item in the concealment mapping table 155-1-j corresponding to the selection patient ID in association.

In step S636, the restoration unit 125-1 writes the concealed data of the selection item of the selection patient ID in the concealed medical record information DB 154-1.

Step S637 is the end of the loop corresponding to step S631. When all the items of the raw medical record information DB 145-1 have been selected, the restoration process is ended. When all the items of the raw medical record information DB 145-1 have not been selected, the control returns to step S631.

Step S638 is the end of the loop corresponding to step S630. When all the patient IDs of the raw medical record information DB 145-1 of the new system have been selected, the restoration process is ended. When all the patient IDs of the raw electronic medical record information DB 145-1 of the new system have not been selected, the control returns to step S530.

According to the information processing system according to the embodiment, in the information processing apparatus of each hospital, since an electronic medical record of a certain format is converted into a common format common for hospitals, when an information analysis agency collects and analyzes electronic medical records from each hospital, it becomes unnecessary for the information analysis agency to arrange the formats, thereby shortening the processing time in the information analysis agency.

According to the information processing system according to the embodiment, since a concealment mapping table associating data before concealment and data after concealment is created, even when the data before concealment is corrupted, the data before concealment can be restored from the data after concealment and the concealment mapping table.

According to the information processing system according to the embodiment, even when the data after concealment is corrupted, the data after concealment can be restored by concealing the data before concealment again.

According to the information processing system according to the embodiment, even when the concealment mapping table is corrupted, the concealment mapping table can be restored by concealing the data before concealment again.

In the information processing system 100 of FIG. 1, the information providing institution may be an institution other than a hospital providing patient medical examination information. For example, the information providing institution may be a shop that provides purchase information of a customer, a school that provides student's score information, an educational institution such as a preparatory school, or a financial institution such as a bank that provides customer's deposit balance, transaction result, etc.

In the case where the information providing institution is a shop, customer purchase information is collected instead of electronic medical record clinical charts, and analysis results indicating customer preferences and the like are provided to an information utilization institution such as a restaurant. In the case where the information providing institution is an educational institution, student score information is collected and analysis results indicating trends and the like for each subject are provided to an information utilization institution such as an educational material production company. In the case where the information providing institution is a financial institution, information such as customer's deposit balance, transaction results, etc. are collected and analysis results indicating the usage situation of loans are provided to an information utilization institution such as a loan company.

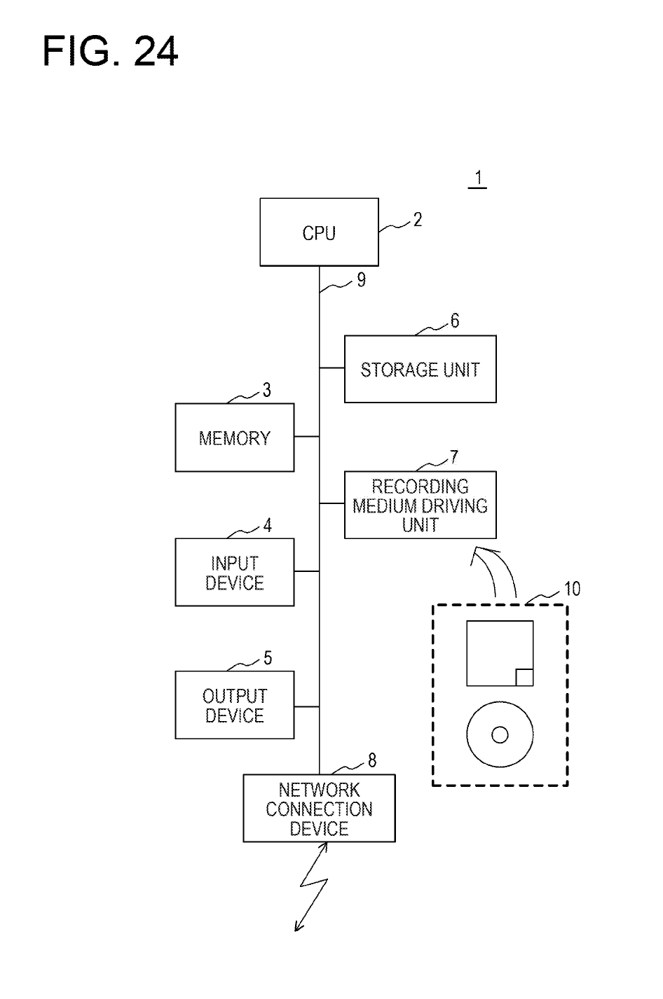

FIG. 24 is a view illustrating the configuration of an information processing apparatus (computer).

The electronic medical record management server 111-i, the doctor PC 161-i, the management PC 171-i, the installer distribution web server 211, the analysis PC 231, the data reading server 221, the data storage server 221 and the user PC 311-i can be realized by the information processing apparatus (computer) 1 as illustrated in FIG. 24, for example.

The information processing apparatus 1 includes a CPU 2, a memory 3, an input device 4, an output device 5, a storage unit 6, a recording medium driving unit 7 and a network connection device 8, which are interconnected by a bus 9.

The CPU 2 is a central processing unit that controls the overall operation of the information processing apparatus 1. The CPU 2 operates as the processing unit 121-1, the web service unit 212 and the concealment logic generation unit 213.

The memory 3 is a memory such as a read only memory (ROM) or a random access memory (RAM) that temporarily stores a program or data stored in the storage unit 6 (or the portable recording medium 10) at the time of execution of the program. The CPU 2 executes the program using the memory 3, thereby executing the various processes described above.

In this case, a program code itself read from the portable recording medium 10 or the like realizes the functions of the embodiment.

The input device 4 is used to input instructions and information from a user or an operator and acquire data used in the information processing apparatus 1. The input device 4 is, for example, a keyboard, a mouse, a touch panel, a camera, a sensor, or the like.

The output device 5 is a device that outputs an inquiry or a processing result to the user or the operator or operates under the control of the CPU 2. The output device 5 is, for example, a display, a printer or the like.

The storage unit 6 is, for example, a magnetic disk device, an optical disk device, a tape device, or the like. The information processing apparatus 1 stores the above-described programs and data in the storage unit 6 and reads and uses them into the memory 3 as necessary. The memory 3 and the storage unit 6 correspond to the storage units 131-1, 214 and 252.

The recording medium driving unit 7 drives the portable recording medium 10 and accesses the recorded contents. The portable recording medium may be any computer-readable recording medium such as a memory card, a flexible disk, a compact disk read only memory (CD-ROM), an optical disk, a magneto-optical disk, or the like. A user stores the above-described program and data in the portable recording medium 10 and reads and uses them into the memory 3 as necessary.

The network connection device 8 is a communication interface which is connected to an arbitrary communication network such as a local area network (LAN) or a wide area network (WAN) and performs data conversion accompanying communication. The network connection device 8 transmits data to a device connected via a communication network or receives data from a device connected via a communication network.

The information processing apparatus 1 may include a portion of all the constituent elements in FIG. 24 and it is also possible to omit some constituent elements depending on usages or conditions.

While the disclosed embodiments and their advantages have been described in detail, those skilled in the art can make various changes, additions and omissions without departing from the spirit and scope of the present disclosure as explicitly set forth in the claims.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to an illustrating of the superiority and inferiority of the invention. Although the embodiments of the present invention have been described in detail, it should be understood that the various changes, substitutions, and alterations could be made hereto without departing from the spirit and scope of the invention.

What is claimed is:

1. An information processing apparatus, comprising:
a memory; and
a processor coupled to the memory and the processor configured to:
convert personal information of a first format, which is stored in a storage device and is used by one of a plurality of information providing institutions, into personal information of a second format used by an analysis device which analyzes personal information collected from the plurality of information providing institutions;
conceal the personal information of the second format to generate concealed personal information of the second format; and
transmit the concealed personal information of the second format to the analysis device.

2. The information processing apparatus according to claim 1, wherein the processor conceals information of a predetermined item among a plurality of items of the personal information of the second format to generate concealed information of the predetermined item of the concealed personal information, and generates mapping information indicating a correspondence relationship between the information of the predetermined item and the concealed information of the predetermined item, and
the storage device stores the personal information of the second format, the concealed personal information and the mapping information,
the processor further configured to:
restore the personal information of the second format based on the concealed personal information and the mapping information when the personal information of the second format stored in the storage device is not able to be read.

3. The information processing apparatus according to claim 2, wherein, when the concealed personal information stored in the storage device is not able to be read, the processor conceals the personal information of the second format again and restores the concealed personal information.

4. The information processing apparatus according to claim 2, wherein, when the mapping information stored in the storage device is not able to be read, the processor conceals the information of the predetermined item of the personal information of the second format again and restores the mapping information.

5. An information processing method, comprising:
converting, by a processor, personal information of a first format, which is stored in a storage device and is used by one of a plurality of information providing institutions, into personal information of a second format used by an analysis device which analyzes personal information collected from the plurality of information providing institutions;
concealing the personal information of the second format to generate concealed personal information of the second format; and
transmitting the concealed personal information of the second format to the analysis device.

6. The information processing method according to claim 5, wherein the act of generating concealed personal information further includes: concealing information of a predetermined item among a plurality of items of the personal information of the second format to generate concealed information of the predetermined item of the concealed personal information, and generating mapping information indicating a correspondence relationship between the information of the predetermined item and the concealed information of the predetermined item, and
the storage device stores the personal information of the second format, the concealed personal information and the mapping information,
the method further comprising:
restoring the personal information of the second format based on the concealed personal information and the mapping information when the personal information of the second format stored in the storage device cannot be read.

7. The information processing method according to claim 6, further comprising:
when the concealed personal information stored in the storage device is not able to be read, concealing the personal information of the second format again and restoring the concealed personal information.

8. The information processing method according to claim 6, further comprising:
when the mapping information stored in the storage device is not able to be read, concealing the information of the predetermined item of the personal information of the second format again and restoring the mapping information.

9. A non-transitory computer-readable recording medium having stored therein a program for causing a computer to execute a process, the process comprising:
converting personal information of a first format, which is stored in a storage device and is used by one of a plurality of information providing institutions, into personal information of a second format used by an analysis device which analyzes personal information collected from the plurality of information providing institutions;
concealing the personal information of the second format to generate concealed personal information of the second format; and
transmitting the concealed personal information of the second format to the analysis device.

10. The non-transitory computer-readable recording medium according to claim 9, wherein the act of generating concealed personal information further includes: concealing information of a predetermined item among a plurality of items of the personal information of the second format to generate concealed information of the predetermined item of the concealed personal information, and generating mapping information indicating a correspondence relationship between the information of the predetermined item and the concealed information of the predetermined item, and
the storage device stores the personal information of the second format, the concealed personal information and the mapping information,
the process further comprising:
restoring the personal information of the second format based on the concealed personal information and the mapping information when the personal information of the second format stored in the storage device cannot be read.

11. The non-transitory computer-readable recording medium according to claim 10, the process further comprising:
when the concealed personal information stored in the storage device is not able to be read, concealing the personal information of the second format again and restoring the concealed personal information.

12. The non-transitory computer-readable recording medium according to claim 10, the process further comprising:
when the mapping information stored in the storage device is not able to be read, concealing the information of the predetermined item of the personal information of the second format again and restoring the mapping information.
